(19) United States
(12) Patent Application Publication
(10) Pub. No.: US 2005/0176755 A1
(43) Pub. Date: Aug. 11, 2005

(54) Aza-bridged-bicyclic amino acid derivatives as alpha4 integrin antagonists

(76) Inventors: Alexey B. Dyatkin, Maple Glen, PA (US); Yong Gong, Warrington, PA (US); Tamara Miskowski, Chalfont, PA (US); Wei He, Audubon, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/976,091

(22) Filed: Oct. 27, 2004

Related U.S. Application Data
(60) Provisional application No. 60/516,574, filed on Oct. 31, 2003.

(51) Int. Cl. C07D 453/04; A61K 31/46; A61K 31/407

(52) U.S. Cl. 514/304; 514/412; 546/135; 548/453

(57) ABSTRACT

The invention is directed to aza-bridged-bicyclic compounds having Formula (I): 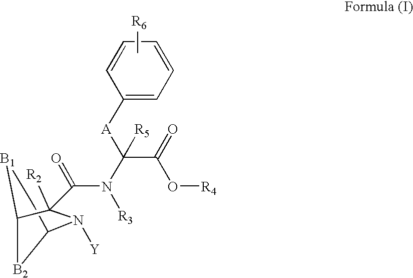 and pharmaceutically acceptable salts thereof. The compounds are useful α4 integrin receptor antagonists and, in particular, α4β1 and α4β7 integrin receptor antagonists. The invention is further directed to methods for use of the instant compounds for treating integrin mediated disorders including, but not limited to, inflammatory disorders, autoimmune disorders and cell-proliferative disorders, methods for preparing the compounds and methods for preparing the intermediates, derivatives and pharmaceutical compositions thereof.

### FIELD OF THE INVENTION

This invention relates to novel compounds and methods for use in treating integrin mediated disorders. More particularly, this invention relates to novel derivatives of aza-bridged-bicyclic amino acid compounds useful as α4 integrin receptor antagonists, methods for treating integrin mediated disorders including, but not limited to, inflammatory, autoimmune and cell-proliferative disorders, methods for preparing the compounds and methods for preparing the intermediates, derivatives and pharmaceutical compositions thereof.

### BACKGROUND OF THE INVENTION

Integrin receptors are transmembrane, non-covalently linked heterodimers consisting of one α-chain and one β-chain. In addition to performing a structural adhesive function, integrin receptors transmit extracellular signals across the plasma membrane. The integrin receptor α₄β₁ (also referred to as VLA-4) mediates cell adhesion by binding with either of two protein ligands: vascular cell adhesion molecule-1 (VCAM-1) (Osborn, L.; et al., Cell, 1989, 59, 1203), or the alternatively-spliced fibronectin variant containing the type III connecting segment (CS-1) (Wayner, E. A.; et al., Cell Biol., 1989, 109, 1321). In contrast to the prototypical integrin receptors α5β1, GPIIb/IIIa and α_{V}β₃ that recognize the Arg-Gly-Asp (RGD) tripeptide sequence in their respective ligands, α_{4β}₁ binds to other primary protein sequences. The α₄β₁ integrin receptor recognizes Gln-Ile-Asp-Ser (QIDS) in VCAM-1 and Ile-Leu-Asp-Val (ILDV) in fibronectin. Although these sequences share a conserved Asp residue with RGD, they are otherwise unrelated. Additionally, recent studies have found that α₄β₁ binds the matrix ligand osteopontin (Bayless, K. J.; et al., J. Cell Sci., 1998, 111, 1165). The osteopontin ligand interaction with the α₄β₁ receptor may be very important as osteopontin is strongly up-regulated in inflammatory settings, including the inflamed lung.

The α₄β₁ integrin receptor is expressed at high levels on mast cells, mononuclear leukocytes, eosinophils, macrophages, and basophils (Adams, S. P.; et al., Ann. Rep. Med. Chem., 1999, 34, 179). The binding of α₄β₁ to cytokine-induced VCAM-1 on high-endothelial venules at sites of inflammation results in leukocyte/endothelium adhesion followed by extravasation into the inflamed tissue (Chuluyan, H. E.; et al., Springer Semin. Immunopathol., 1995, 16, 391). The role of mast cells and eosinophils in lung inflammation is well-established. Induction of VCAM-1 expression on airway endothelial cells seems to play a central role in lung inflammation. The α4β1 receptor interaction with VCAM-1 also exerts an important effect in stem cell adhesion to bone marrow stromal cells (Simmons, P. J.; et al., Blood, 1992, 80, 388).

The α₄β₇ integrin is expressed at high levels on lymphocytes and T cells. The trafficking of lymphocytes from the vasculature to normal mucosa and lymphoid tissues is mediated by adhesion of mucosal addressing cell adhesion molecule-1 (MAdCAM-1) with the integrin receptor α₄β₇. In an inflammatory setting, MAdCAM-1, an immunoglobulin superfamily adhesion molecule, specifically binds α4β7-expressing lymphocytes and participates in the homing of these cells to the mucosal endothelium. Cloning studies of human MAdCAM-1 have shown that the Leu-Asp-Thr-Ser-Leu (LDTSL) sequence of the CD loop is conserved. In fact, LDT-based peptides bind to α₄β₇ in a MAdCAM-1/RPMI-8866 cell adhesion assay with IC₅₀ values in the 1-10 uM range (Shroff, H. N.; et al., Bioorg. Med. Chem. Lett., 1998, 8, 1601).

The extensive biology mediated by integrins in general and compelling data for the pathophysiological role of the leukocyte cell adhesion receptor α4β1 have spurred interest in the study of α4β1 antagonists in vivo. Cellular adhesion and migration mediated through the β1 integrins are critical components of cellular recruitment processes. The integrin α4β1 provides a key co-stimulatory signal supporting cell activation leading to growth factor and cytokine production and mediator release. Through recognition of the extracellular matrix, α4β1 increases the survival of activated cells by inhibiting apoptosis (Yoshikawa, H.; et al., J. Immunol., 1996, 156, 1832).

Monoclonal antibodies directed against α4β1 or VCAM-1 have been shown to be effective modulators in animal models of chronic inflammatory diseases such as asthma (Laberge, S.; et al., Am. J. Respir. Crit. Care Med., 1995, 151, 822), rheumatoid arthritis (Barbadillo, C.; et al., Springer Semin. Immunopathol., 1995, 16, 375) and inflammatory bowel disease (Powrie, F.; et al., Ther. Immunol., 1995, 2, 115). The initial research in the low molecular weight α4β1 antagonist arena has focused on simple linear analogues of the prototype Leu-Asp-Val sequence. Phenylacetyl-Leu-Asp-Phe-D-Pro-NH₂ (having an α4β1 IC₅₀ value of 2 uM) exhibited efficacy similar to the α4 antibody PS/2 in a mouse model of oxazolone-induced contact hypersensitivity when administered at 6 mg/kg, sc (Tamraz, S.; et al., Springer Semin. Immunopathol. 1995, 16, 437). This tetrapeptide was also effective in a hyperlipidemic rabbit heterotopic heart allograft model (Molossi, S.; et al., J. Clin. Invest. 1995, 95, 2601).

Animal models of asthma have shown that the peptide antagonist BIO-1211 inhibits eosinophilia and airway hyper responsiveness (Lin, K-C.; et al., J. Med. Chem. 1999, 42, 920). Pre-treatment of allergic sheep with a 3 mg nebulized dose of BIO-1211 (having an α4β1 IC₅₀ value of 1 nM; 1000-fold selective over α4β7 ) inhibited early and late airway responses following antigen challenge and prevented development of nonspecific airway hyperresponsiveness to carbachol. These results suggest that compounds like BIO-1211 can effect broad pleiotropic activities by acting at α4β1 to achieve pronounced efficacy similar to corticosteroids.

VLA-4 antagonism may also be effective in reducing restenosis following percutaneous coronary interventions. Administration of an anti-α4 antibody attenuated smooth muscle cell migration associated with electrical injury of rabbit carotid arteries (Kling D, Fingerle J, Harlan J M, Lobb, R R and Lang, F, Mononuclear leukocytes invade rabbit arterial intima during thickening formation via CD-18 and VLA-4-dependent mechanisms and stimulate smooth muscle migration, Circ. Res., 1995, 77, 1121-1128) and was shown to reduce neointimal formation in baboon carotid arteries following endarterectomy (Lumsden A B, Chen C, Hughes J D, Kelly A B, Hanson S and Harker L, Anti-VLA4 antibody reduces intimal hyperplasia in the endarterectomized carotid artery in non-human primates, J. Vasc. Surg., 1997, 26, 87-93). Furthermore, treatment with z anti-α4 antibody was associated with less neoadventitia formation and less lumenal narrowing 14 days after balloon injury of porcine coronary arteries (Labinez M, Hoffert C, pels K, Aggarawal S, Pepinsky R B, Leonw D, Koteliansky V, Lobb, RR and O'Brien E O, Infusion on and anti-alpha4 integrin antibody is associated with less adventitial formation after balloon injury of porcine coronary arteries, Can. J. Cardiol., 2000, 16,187-196).

The recruitment of leukocytes, particularly monocytes to the vessel wall is a key component in the development of atherosclerotic lesions. VCAM-1 expression has been reported on endothelial cells in atherosclerotic lesions in humans (O'Brien K D, Allen M D, McDonald T O, Chait A, Harlan J M, Fishbein D, McCarty J, Ferguson M, Hudkins K, Benjamin C D, et al., Vascular cell adhesion molecule-1 is expressed in human atherosclerotic plaques: implications for the mode of progression of advanced atherosclerosis, J. Clin Invest., 1993, 92, 945-951), mice (Nakahima Y, Raines E W, Plump A S, Breslow J L and Ross R, Upregulation of VCAM-1 and ICAM-1 at atherosclerotic-prone sites on the endothelium of ApoE-deficient mouse, Arterioscier. Thromb. Vasc. Biol., 1998, 18, 842-851) and rabbits (Ilyama K, Hajra L, Iiyam M, Li, H, DiChura M, Medoff B D and Cybulsky M I, Patterns of vascular cell adhesion molecule-1 and intercellular adhesion molecule-1 expression in rabbit and mouse atherosclerotic lesion and at sites predisposed to lesion formation, Circ. Res., 1999, 85, 199-207). Furthermore, a synthetic peptidomimetic of the connecting segment-1 (CS-1) which blocks α₄β₁ on the leukocyte demonstrated reduced leukocyte homing and lipid accumulation in the aortic sinus in both wild type mice and mice with a low density lipoprotein null mutation (LDLR −/−) maintained on a high fat diet (Shih P T, Brennan M-L, Vora D K, Territo M C, Strahl D, Elices M J, Aldons J and Berliner J A, Blocking very late antigen-4 integrin decreases leukocyte entry and fatty streak formation in mice fed an atherogenic diet, Circ. Res., 1999, 84, 345-351). In studies using isolated carotid arteries from ApoE −/− mice (these mice develop spontaneous arterial atherosclerotic lesions with advanced lesions similar to those observed in humans), administration on blocking antibodies to VCAM-1 inhibited the majority of adhesion of monocytes or U937 cells on early atherosclerotic endothelia. In addition, a peptide which inhibits binding of α4β1 to both VCAM-1 and fibronectin was also effective in this model (Huo Y, Hafez-Moghadem A and Ley K, Role of vascular cell adhesion molecule-1 and fibronectin connecting segment-1 in monocyte rolling and adhesion on early atherosclerotic lesions, Circ. Res., 2000, 87, 153-159). These data support the role of a₄β₁ in regulating leukocyte recruitment in early and advanced atherosclerotic lesions.

Antibodies to MAdCAM-1 or integrin α4β7 inhibit lymphocyte binding to affinity-purified MAdCAM-1 or MAdCAM-1 transfectants in vitro (Hamann, A.; et al., J. Immunol. 1994, 152, 3282). The antibodies also block localization of lymphocytes to Peyer's patches. Murine MAdCAM-1 recognizes only α4β7 positive human lymphocyte cells lines and α4β7-high memory T cells. An in vivo role of α4β7 in inflammation has been suggested by increased expression of MAdCAM-1 on HEV-type vessels in the chronically inflamed pancreas of the non-obese mouse (Hanninen, A. C.; et al., J. Clin. Invest. 1993, 92, 2509). In fact, animal models underscore a significant function of α4β7 in both colitis (Fong, S.; et al., Immunol. Res. 1997, 16, 299) and lymphocytic inflammation of pancreatic islets or development of diabetes (Yang, X.; et al., Diabetes 1997, 46, 1542).

PCT application WO 98/53814 describes heterocyclic amide compounds as antagonists for VLA-4 and/or α4β₇ antagonists of the formula: 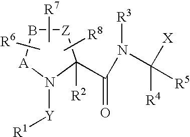
or a pharmaceutically acceptable salt thereof wherein
R¹ is alkyl, alkenyl, alkynyl, C_{y}, C_{y}-alkyl, C_{y}-alkenyl or C_{y}-alkynyl; wherein alkyl, alkenyl and alkynyl are optionally substituted with R^{a}; and C_{y} is optionally substituted with R^{b}; R² is hydrogen, alkyl, alkenyl, alkynyl, aryl, aralkyl, heteroaryl or heteroaralkyl; wherein alkyl, alkenyl and alkynyl are optionally substituted with R^{a}; and aryl and heteroaryl are optionally substituted with R^{b}; R³ is hydrogen, alkyl, C_{y} or C_{y}-alkyl; wherein alkyl is optionally substituted with R^{a}; and C_{y} is optionally substituted with R^{b}; R⁴ is hydrogen, alkyl, alkenyl, alkynyl, C_{y}, C_{y}-alkyl, C_{y}-alkenyl or C_{y}-alkynyl; wherein alkyl, alkenyl and alkynyl are optionally substituted with phenyl and R^{x}; and C_{y} is optionally substituted with R^{y}; or, R³, R⁴ and the atoms to which they are attached together form a mono- or bicyclic ring containing 0-2 additional heteroatoms selected from N, O and S; R⁵ is hydrogen, alkyl, alkenyl, alkynyl, aryl, aralkyl, heteroaryl or heteroaralkyl; wherein alkyl, alkenyl and alkynyl are optionally substituted with R^{x}; and aryl and heteroaryl are optionally substituted with R^{y}; or, R⁴, R⁵ and the carbon to which they are attached together form a 3-7 membered mono- or bicyclic ring containing 0-2 heteroatoms selected from N, O and S; R⁶, R⁷ and R⁸ are each independently selected from R^{d} or R^{x}; or, two of R⁶, R⁷ and R⁸ and the atom to which both are attached, or two of R⁶, R⁷ and R⁸ and the two adjacent atoms to which they are attached together form a 5-7 membered saturated or unsaturated monocyclic ring containing 0-3 heteroatoms selected from N, O or S; R^{a} is C_{y}, or a group selected from R^{x}; wherein C_{y} is optionally substituted with R^{c}; R^{b} is a group selected from R^{a}, alkyl, alkenyl, alkynyl, aralkyl or heteroaralkyl; wherein alkyl, alkenyl, alkynyl, aryl and heteroaryl are optionally substituted with R^{c}; R^{b} is halogen, NO₂, C(O)OR^{f}, alkyl, alkoxy, aryl, aralkyl, aryloxy, heteroaryl, NR^{f}R^{g}, NR^{f}C(O)R^{g}, NR^{f}C(O)NR^{f}R^{g}, or CN; R^{d} and R^{e} are independently selected from hydrogen, alkyl, alkenyl, alkynyl, C_{y} and C_{y}-alkyl; wherein alkyl, alkenyl, alkynyl and C_{y} are optionally substituted with R^{c}; or, R^{d} and R^{e} together with the atoms to which they are attached form a heterocyclic ring of 5-7 members containing 0-2 additional heteroatoms independently selected from N, O and S; R^{f} and R^{g} are independently selected from hydrogen, alkyl, C_{y} and C_{y}-alkyl; wherein C_{y} is optionally substituted with alkyl; or, R^{f}; and R^{g} together with the carbon to which they are attached form a ring of 5-7 members containing 0-2 heteroatoms independently selected from N, O and S; R^{h} is hydrogen, alkyl, alkenyl, alkynyl, cyano, aryl, aralkyl, heteroaryl, heteroaralkyl or —SO₂Rⁱ; wherein alkyl, alkenyl and alkynyl are optionally substituted with R^{a}; and aryl and heteroaryl are optionally substituted with R^{b}; Rⁱ is alkyl, alkenyl, alkynyl or aryl; wherein alkyl, alkenyl, alkynyl and aryl are each optionally substituted with R^{c}; R^{k} is —OR^{d}, —NO₂, halogen, —S(O)ₘR^{d}, —SR^{d}, —S(O₂)OR^{d}, —S(O)ₘNR^{d}R^{e}, —NR^{d}R^{e}, —O(CR^{f}R^{g})ₙNR^{d}R^{e}, —C(O)R^{d}, —CO₂R^{d}, —CO₂(C R^{f}R^{g})ₙC(O)NR^{d}R^{e}, —OC(O)R^{d}, CN, C(O)NR^{d}R^{e}, —NR^{d}C(O)R^{e}, —OC(O)NR^{d}R^{e}, —NR^{d}C(O)OR^{e}, —NR^{d}C(O)NR^{d}R^{e}, —CR^{d}(N—OR^{e}), —CF₃, oxo, —NR^{d}C(O)NR^{d}SO₂Rⁱ, —NR^{d}S(O)ₘR^{e}, —OS(O₂)OR^{d} or —OP(O)(OR^{d})₂; R^{y} is a group selected from R^{k}, alkyl, alkenyl, alkynyl, aralkyl, heteroaralkyl, cycloalkyl or heterocyclyl; wherein alkyl, alkenyl, alkynyl and aryl are each optionally substituted with R^{x}; C_{y} is cycloalkyl, heterocyclyl, aryl or heteroaryl; m is an integer from 1 to 2; n is an integer from 1 to 10; X is —C(O)OR^{d}, —P(O)(OR^{d})(OR^{e}), —P(O)(R^{d})(OR^{e}), S(O)ₘOR^{d}, C(O)NR^{d}R^{h} or -5-tetrazolyl; Y is —C(O)—, —O—C(O)—, —NR^{e}C(O)—, —SO₂—, —P(O)(OR⁴) or C(O)C(O); Z and A are independently selected from —C— and —C—C—; and, B is selected from the group consisting of a bond, —C—, —C═C—, —C═C—, a heteroatom selected from the group consisting of N, O and S; and —S(O)ₘ—. Specific examples which exemplify some of the typical compounds disclosed have the following typical formulae:

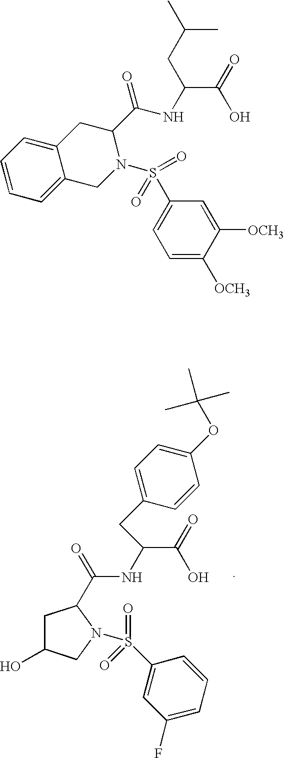

PCT application WO 00/43354 describes multicyclic compounds as inhibitors of leukocyte adhesion mediated by VLA-4 of the formula: 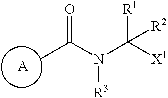
wherein ring A is a multicyclic bridged cycloalkyl, multicyclic bridged cycloalkenyl or multicyclic bridged heterocyclic group provided the multicyclic bridged heterocyclic group does not contain a lactam and further wherein said multicyclic bridged cycloalkyl, multicyclic bridged cycloalkenyl or multicyclic bridged heterocyclic group is optionally substituted, on any ring atom capable of substitution, with 1-3 substituents selected from the group consisting of alkyl, substituted alkyl, alkoxy, substituted alkoxy, acyl, acylamino, thiocarbonyl-amino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂— substituted aryl, —OS(O)₂-heteroaryl, —OS(O)₂-substituted heteroaryl, —OS(O)₂— heterocyclic, —OS(O)₂-substituted heterocyclic, —OSO₂—NRR where each R is independently hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂— heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, —N[S(O)₂—R′]₂ And —N[S(O)₂—NR′]₂ where each R′ is independently selected from the group consisting of alkyl, substituted alkyl, aryl, substituted aryl, heteroaryl, substituted heteroaryl, heterocyclic and substituted heterocyclic, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl; R¹ is selected from the group consisting of: (a) —(CH₂)ₓ—Ar—R⁵ where R⁵ is selected from the group consisting of —O-Z-NR⁶R⁶ and —O-Z-R⁷ wherein R⁶ and R^{6′} are independently selected from the group consisting of hydrogen, alkyl, substituted alkyl, cycloalkyl, substituted cycloalkyl, heterocyclic, substituted heterocyclic, and where R⁶ and R⁶′ are joined to form a heterocycle or a substituted heterocycle, R⁷ is selected from the group consisting of heterocycle and substituted heterocycle, and Z is selected from the group consisting of —C(O)— and —SO₂—, Ar is aryl, heteroaryl, substituted aryl or substituted heteroaryl, x is an integer of from 1 to 4; (b) Ar¹-Ar²-C₁₋₁₀alkyl-, Ar¹-Ar²-C₂₋₁₀alkenyl-, Ar¹-Ar²-C₂₋₁₀alkynyl-, wherein Ar¹ and Ar² are independently aryl or heteroaryl each of which is optionally substituted with one to four substituents independently selected from R^{b}; alkyl, alkenyl and alkynyl are optionally substituted with one to four substituents independently selected from R^{a}; (c)-(CH₂)ₓ—Ar—R⁸, wherein R⁸ is heterocyclic or substituted heterocyclic; Ar is aryl, heteroaryl, substituted aryl or substituted heteroaryl, x is an integer of from 1 to 4; (d)-(CH₂)ₓ—Ar—R⁹, wherein R⁹ is —C₁₋₁₀alkyl, —C₂₋₁₀alkenyl or —C₂₋₁₀alkynyl, wherein alkyl, alkenyl and alkynyl are optionally substituted with one to four substituents selected from R^{a}; Ar is aryl, heteroaryl, substituted aryl or substituted heteroaryl, x is an integer of from 1 to 4; (e)-(CH₂)ₓ-Cy-, wherein Cy is optionally substituted with 1 to 4 substituents selected from R² is selected from the group consisting of hydrogen, C₁₋₁₀alkyl, C₂₋₁₀alkenyl, C₂₋₁₀alkynyl, aryl, aryl C₁₋₁₀alkyl, heteroaryl, and heteroaryl C₁₋₁₀alkyl, wherein alkyl, alkenyl and alkynyl are optionally substituted with one to four substituents selected from R^{a}, and aryl and heteroaryl are optionally substituted with one to four substituents independently selected from R^{b}; R³ is selected from the group consisting of hydrogen, C₁₋₁₀ alkyl optionally substituted with one to four substituents independently selected from R^{a} and Cy optionally substituted with one to four substituents independently selected from R^{b}; R^{a} is selected from the group consisting of Cy, —OR^{d}, —NO₂, halogen, —S(O)ₘR^{d}, —SR^{d}, —S(O)₂OR^{d}, —S(O)ₘNR^{d}R^{e}, NR^{d}R^{e}, —O(CNR^{f}R^{g})ₙNR^{d}R^{e}, —C(O)R^{d}, —CO₂R^{d}, —CO₂(CR^{f}R^{g})ₙCONR^{d}R^{e}, —OC(O)R^{d}, —CN, C(O)NR^{d}R^{e}, NR^{d}C(O)R^{e}, —OC(O)NR R^{e}, —NR^{d}C(O)OR^{e}, —NR^{d}C(O)NR^{d}R^{e}, —CR^{d}(N—OR^{e}), CF₃, and —OCF₃; wherein Cy is optionally substituted with one to four substituents independently selected from R^{c}; R^{b} is selected from the group consisting of R^{a}, C₁₋₁₀alkyl, C₂₋₁₀alkenyl, C₂₋₁₀alkynyl, aryl C₁₋₁₀alkyl, heteroaryl, C₁₋₁₀alkyl, wherein alkyl, alkenyl, aryl, heteroaryl are optionally substituted with a group independently selected from R^{c}; R^{c} is selected from the group consisting of halogen, amino, carboxy, C₁₋₄alkyl, C₁₋₄alkoxy, aryl, aryl C₁₋₄alkyl, hydroxy, CF₃, and aryloxy; R^{d} and R^{e} are independently selected from hydrogen, C₁₋₁₀alkyl, C₂₋₁₀alkenyl, C₂₋₁₀alkynyl, Cy and Cy-C₁₋₁₀alkyl, wherein alkyl, alkenyl, alkynyl and Cy are optionally substituted with one to four substituents independently selected from R^{c}; or R^{d} and R^{e} together with the atoms to which they are attached form a heterocyclic ring of 5 to 7 members containing 0-2 additional heteroatoms independently selected from oxygen, sulfur and nitrogen; R^{f} and R^{g} are independently selected from hydrogen, C₁₋₁₀alkyl, Cy and Cy-C₁₋₁₀alkyl; or R^{a} Rf and R^{a} R9 together with the carbon to which they are attached form a ring of 5 to 7 members containing 0-2 heteroatoms independently selected from oxygen, sulfur and nitrogen; R^{h} is selected from the group consisting of hydrogen, C₁₋₁₀alkyl, C₂₋₁₀alkenyl, C₂₋₁₀alkynyl, cyano, aryl, aryl C₁₋₁₀alkyl, heteroaryl, heteroaryl C₁₋₁₀alkyl, or —SO₂Rⁱ; wherein alkyl, alkenyl, and alkynyl are optionally substituted with one to four substitutents independently selected from R^{a}; and aryl and heteroaryl are each optionally substituted with one to four substituents independently selected from R^{b}; Rⁱ is selected from the group consisting of C₁₋₁₀alkyl, C₂₋₁₀alkenyl, C₂₋₁₀alkynyl, and aryl; wherein alkyl, alkenyl, alkynyl and aryl are each optionally substituted with one to four substituents independently selected from R^{c}; Cy is cycloalkyl, heterocyclyl, aryl, or heteroaryl; X¹ is selected from the group consisting of —C(O)OR^{d}, —P(O)(OR^{d})(OR^{e}), —P(O)(R^{d})(OR^{e}), —S(O)ₘOR^{d}, —C(O)NR^{d}R^{h}, and -5-tetrazolyl; m is an integer from 1 to 2; n is an integer from 1 to 10; and pharmaceutically acceptable salts thereof. Preferred compounds of this invention are represented by formula II: 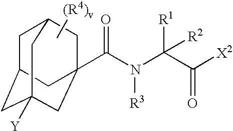
wherein R¹, R² and R³ are as defined above; Y is selected from the group consisting of hydrogen, R^{d}, Cy, —OR^{d}, —NO₂, halogen, —S(O)ₘR^{d}, —SR^{d}, —S(O)₂OR^{d}, —S(O)ₘNR^{d}R^{e}, —NR^{d}R^{e}, —O(CR^{f}R^{g})ₙNR^{d}R^{e}, —C(O)R^{d}, —CH(OH)R^{d}, —CO₂R^{d}, —CO₂(CR^{f}R)ₙCONR^{d}R^{e}, —OC(O)R^{d}, —CN, C(O)NR^{d}R^{e}, NR^{d}C(O)R^{e}, OC(O)NR^{d}R^{e}, —NR^{d}C(O)OR^{e}, —NR^{d}C(O)NR^{d}R^{e}, —CR^{d}(N—OR^{e}), CF₃, and —OCF₃; wherein Cy is optionally substituted with one to four substituents independently selected from R^{c}; where Cy, R^{c}, R^{d}, R^{e}, R^{f}, R^{g}, R^{h}, m and n are as defined herein; R⁴ is selected from the group consisting of alkyl, substituted alkyl, alkoxy, substituted alkoxy, acyl, acylamino, thiocarbonyl-amino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, carboxyl, carboxylalkyl, carboxyl-substituted alkyl, carboxyl-cycloalkyl, carboxyl-substituted cycloalkyl, carboxylaryl, carboxyl-substituted aryl, carboxyllieteroaryl, carboxyl-substituted heteroaryl, carboxyllieterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thiolheteroaryl, substituted thiol heteroaryl, thiolheterocyclic, substituted thiolheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where each R is independently hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, —N[S(O)₂—R′]₂ and —N[S(O)₂—NR′]₂ where each R′ is independently selected from the group consisting of alkyl, substituted alkyl, aryl, substituted aryl, heteroaryl, substituted heteroaryl, heterocyclic and substituted heterocyclic, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl; or R^{b} where R^{b} is as defined above; X² is selected from the group consisting of hydroxyl, alkoxy, substituted alkoxy, alkenoxy, substituted alkenoxy, cycloalkoxy, substituted cycloalkoxy, cycloalkenoxy, substituted cycloalkenoxy, aryloxy, substituted aryloxy, heteroaryloxy, substituted heteroaryloxy, heterocyclyloxy, substituted heterocyclyloxy and —NR″R″ where each R″ is independently selected from the group consisting of hydrogen, alkyl, substituted alkyl, alkenyl, substituted alkenyl, cycloalkyl, substituted cycloalkyl, aryl, substituted aryl, heteroaryl, substituted heteroaryl, heterocyclic and substituted heterocyclic; or R^{d} where R^{d} is as defined above; v is an integer ranging from 0 to 3; and pharmaceutically acceptable salts thereof.

The structural topology represented by the formulae described in these references differs significantly from that represented by the compounds of the present invention.

Accordingly, it is an object of the present invention to provide aza-bridged-bicyclic compounds that are α4 integrin receptor antagonists; more particularly, the α₄β₁ and the α₄β₇ integrin receptor. It is also an object of the present invention to provide a process for preparing derivatives of aza-bridged-bicyclic amino acid compounds, compositions, intermediates and derivatives thereof. It is a further object of the invention to provide methods for the treatment of integrin mediated disorders that are ameliorated by inhibition of the α₄β₁ and α₄β₇ integrin receptor including, but not limited to, inflammatory, autoimmune and cell-proliferative disorders.

### SUMMARY OF THE INVENTION

The present invention is directed to aza-bridged-bicyclic compounds having Formula (I): 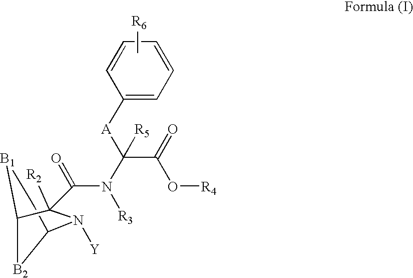
wherein
Y is selected from the group consisting of hydrogen, —C(O)(CH₂)₀₋₄R₁₈, —C(O)(CH₂)_{q}NC(O)R₁, —C(O)(CH₂)_{q}SR₁, —C(O)(CH₂)_{q}SOR₁; and —C(O)(CH₂)_{q}SO₂R₁;
q is an integer from 1 to 8;
R₁ is selected from the group consisting of hydrogen, R₇ and R₈;
R₂, R₃, and R₅ are independently selected from the group consisting of hydrogen and C₁₋₈alkyl; wherein C₁₋₈alkyl is optionally substituted with one to three substituents independently selected from OH, halogen, C₁₋₈ alkoxy, carboxy, amino, N-(C₁₋₈alkyl)amino, N,N-(C₁₋₈ dialkyl)amino, CF₃, OCF₃ and R₉; provided that R₃ additionally may be a bond when forming a monocyclic ring;
R₄ is selected from the group consisting of hydrogen, C₁₋₄alkyl and N,N-C₁₋₄ glycolamide; wherein C₁₋₄alkyl is optionally substituted with one to three substituents independently selected from OH, halogen, C₁₋₈ alkoxy, carboxy, amino, N-(C₁₋₈alkyl)amino, N,N-(C₁₋₈ dialkyl)amino, CF₃, OCF₃ and R₉; provided that R₄ additionally may be a bond when forming a monocyclic ring;
Note: when R₉ is amended to include OH that means the C₁₋₄alkyl can now be substituted with hydroxy, which means we do not need to add (CH₂CH₂)ₙOH.
wherein R₃, and R₄ may form a monocyclic ring;
   when R₃ and R₄ comprise a bond and C₁₋₈alkyl or optionally when both R₃ and R₄ are C₁₋₈alkyl, R₃ and R₄ together with the atoms to which each is attached will form a five to seven membered monocyclic ring optionally containing one to two additional heteroatoms independently selected from the group consisting of N, O and S;
R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀, —N(R₁₁)SO₂—R₁₂, —N(R₁₁)C(O)—N(R₁₁,R₁₀), —N(R₁₁)C(O)—N(R₁₁,R₁₂), —N(R₁₁)C(O)—N(R₁₂,R₁₇), —C(O)—N(R₁₁,R₁₀), —C(O)—N(R₁₁,R₁₂), —C(O)—N(R₁₂,R₁₇); —OC(O)—N(R₁₁,R₁₀), —OC(O)—N(R₁₁,R₁₂), —OC(O)—N(R₁₂,R₁₇), —OC(O)—R₁₀, —OC(O)—R₁₂, —O—R₁₀ and R₁₀-(C₁₋₈)alkoxy;
R₇, R₉ R₁₀ and R₁₄ are independently selected from the group consisting of cycloalkyl, heterocyclyl, aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
R₈ is selected from the group consisting of C₁₋₈alkyl, C₂₋₈alkenyl, C₂₋₈alkynyl, C₁₋₈alkoxy and (halo)₁₋₃(C₁₋₈)alkyl; wherein C₁₋₈alkyl, C₂₋₈alkenyl, C₂₋₈alkynyl and C₁₋₈alkoxy are optionally substituted with one to three substituents independently selected from R₁₄;
R₁₂, R₁₃, R₁₇ and R₁₉ are independently selected from the group consisting of C₁₋₈alkyl, C₂₋₈alkenyl, C₂₋₈alkynyl, and (halo)₁₋₃(C₁₋₈)alkyl; wherein C₁₋₈alkyl, C₂₋₈alkenyl and C₂₋₈alkynyl are optionally substituted with one to three substituents independently selected from R₁₄;
R₁₁ is selected from the group consisting of hydrogen and C₁₋₈alkyl;
R₁₈ is selected from the group consisting of hydroxy, C₁₋₈alkoxy, C₁₋₈alkyloxy C₁₋₈alkyl, C₁₋₈alkoxycarbonylamino C₁₋₈alkyl; amino, amino C₁₋₈alkyl, C₁₋₈alkyl amino C₁₋₈alkyl, diC₁₋₈alkylamino C₁₋₈alkyl, benzo-fused heterocyclyl, polycycloalkyl and hydroxy C₁₋₈alkyl; wherein the benzo-fused heterocyclyl is substituted with C(O)R₁₉ and C(O)OR₁₉;
A is C₁₋₂alkylene optionally substituted with one to two substituents independently selected from R₁₃;
when R₃ is C₁₋₈alkyl, optionally A and R₃ together with the atoms to which each is attached may form a five to seven membered monocyclic ring optionally containing one to two additional heteroatoms independently selected from the group consisting of N, O and S;
when R₄ is C₁₋₈alkyl, optionally A and R₄ together with the atoms which each is attached may form a five to seven membered monocyclic ring optionally containing one additional heteroatom selected from the group consisting of N, O and S;
when R₅ is C₁₋₈alkyl, optionally A and R₅ together with the atoms which each is attached may form a three to seven membered monocyclic ring optionally containing one to two heteroatoms independently selected from the group consisting of N, O and S; and,
B₁ and B₂ are independently selected from the group consisting of C₁₋₂alkylene and C₂alkenylene optionally substituted with one to two substituents independently selected from the group consisting of halogen, hydroxy, hydroxy(C₁₋₈)alkyl, hydroxy(C₁₋₈)alkoxy, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
   and pharmaceutically acceptable salts, racemic mixtures, diastereomers and enantiomers thereof.

An embodiment of the present invention is directed to aza-bridged-bicyclic compounds having Formula (II): 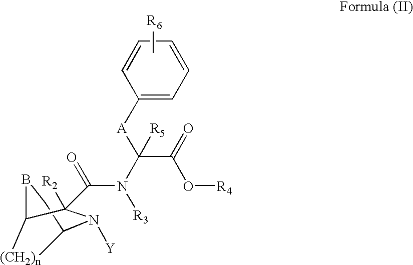
wherein
Y is selected from the group consisting of hydrogen, C(O)(CH₂)₀₋₄R₁₈, —C(O)(CH₂)_{q}NC(O)R₁, —C(O)(CH₂)_{q}SR₁, —C(O)(CH₂)_{q}SOR₁; and —C(O)(CH₂)_{q}SO₂R₁;
q is an integer from 1 to 4;
R₁ is selected from the group consisting of hydrogen, R₇ and R₈;
R₂, R₃, and R₅ are independently selected from the group consisting of a bond, hydrogen and C₁₋₈alkyl; wherein C₁₋₈alkyl is optionally substituted with one to three substituents independently selected from R₉; provided that R₃, can only be a bond when forming a monocylic ring
R₄ is selected from the group consisting of hydrogen, C₁₋₄alkyl and N,N-(C₁₋₄ glycolamide; wherein C₁₋₄alkyl is optionally substituted with one to three substituents independently selected from OH, halogen, C₁₋₈ alkoxy, carboxy, amino, N-(C₁₋₈alkyl)amino, N,N-(C₁₋₈ dialkyl)amino, CF₃, OCF₃ and R₉; provided that R₄ additionally may be a bond when forming a monocyclic ring;
wherein R₃ and R₄ may form a monocyclic ring:
   when R₃ and R₄ comprise a bond and C₁₋₈alkyl or optionally when both R₃ and R₄ are C₁₋₈alkyl, R₃ and R₄ together with the atoms to which each are attached form a five to seven membered monocyclic ring optionally containing one to two additional heteroatoms independently selected from the group consisting of N, O and S;
R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀, —N(R₁₁)SO₂—R₁₂, —N(R₁₁)C(O)—N(R₁₁,R₁₀), —N(R₁₁)C(O)—N(R₁₁,R₁₂), —N(R₁₁)C(O)—N(R₁₂,R₁₇), —C(O)—N(R₁₁,R₁₀), —C(O)—N(R₁₁,R₁₂), —C(O)—N(R₁₂,R₁₇), —OC(O)—N(R₁₁,R₁₀), —OC(O)—N(R₁₁,R₁₂), —OC(O)—N(R₁₂,R₁₇), —OC(O)—R₁₀, —OC(O)—R₁₂, —O—R₁₀ and R₁₀-(C₁₋₈)alkoxy;
R₇ R₉, R₁₀ and R₁₄ are independently selected from the group consisting of cycloalkyl, heterocyclyl, aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl; C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
R₈ is independently selected from the group consisting of C₁₋₈alkyl, C₂₋₈alkenyl, C₂₋₈alkynyl, C₁₋₈alkoxy and (halo)₁₋₃(C₁₋₈)alkyl; wherein C₁₋₈alkyl, C₂₋₈alkenyl, C₂₋₈alkynyl and C₁₋₈alkoxy are optionally substituted with one to three substituents independently selected from R₁₄;
R₁₂, R₁₃, R₁₇, and R₁₉ are independently selected from the group consisting of C₁₋₈alkyl, C₂₋₈alkenyl, C₂₋₈alkynyl and (halo)₁₋₃(C₁₋₈)alkyl; wherein C₁₋₈alkyl, C₂₋₈alkenyl and C₂₋₈alkynyl are optionally substituted with one to three substituents independently selected from R₁₄;
R₁₁ is selected from the group consisting of hydrogen and C₁₋₈alkyl;
R₁₈ is selected from the group consisting of hydroxy, C₁₋₈alkoxy, C₁₋₈ alkyloxyC₁₋₈alkyl, C₁₋₈ alkylcarbonyl aminoC₁₋₈ alkyl, amino, aminoC₁₋₈alkyl, C₁₋₈alkyl amino C₁₋₈alkyl, diC₁₋₈alkylamino C₁₋₈alkyl, benzo-fused heterocyclyl, polycycloalkyl and hydroxy C₁₋₈alkyl; wherein the benzo-fused heterocyclyl is substituted with C(O)R₁₉ and C(O)OR₁₉.
A is C₁₋₂alkylene optionally substituted with one to two substituents independently selected from R₁₃;
when R₃ is C₁₋₈alkyl-optionally A and R₃ together with the atoms to which each is attached form a five to seven membered monocyclic ring optionally containing one to two additional heteroatoms independently selected from the group consisting of N, O and S;
when R₄ is C₁₋₈alkyl, optionally A and R₄ together with the atoms to which each is attached form a five to seven membered monocyclic ring optionally containing one additional heteroatom selected from the group consisting of N, O and S;
when R₅ is C₁₋₈alkyl, optionally A and R₃ together with the atoms to which each is attached form a three to seven membered monocyclic ring optionally containing one to two heteroatoms independently selected from the group consisting of N, O and S;
B is selected from the group consisting of C₁₋₂alkylene and C₂alkenylene optionally substituted with one to two substituents independently selected from the group consisting of halogen, hydroxy, hydroxy(C₁₋₈)alkyl, hydroxy(C₁₋₈)alkoxy, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃; and,
n is an integer from 1 to 2;
   and pharmaceutically acceptable salts, racemic mixtures, diastereomers and enantiomers thereof.

An embodiment of the present invention is also directed to a process for preparing the instant aza-bridged-bicyclic compounds, compositions, intermediates and derivatives thereof. Another embodiment of the present invention is directed to pharmaceutical compositions comprising the compounds of the present invention.

The aza-bridged-bicyclic amino acid derivatives of the present invention are useful α4 integrin receptor antagonists and, more particularly, α₄β₁ and α₄β₇ integrin receptor antagonists. A further embodiment of the present invention is directed to a method for the treatment of integrin mediated disorders that are ameliorated by inhibition of the α₄β₁ and α₄β₇ integrin receptor including, but not limited to, inflammatory, autoimmune and cell-proliferative disorders. In an illustration of the invention, the inflammatory, autoimmune and cell-proliferative disorders include, but are not limited to, inflammation and autoimmunity, asthma and bronchoconstriction, restenosis and atherosclerosis, psoriasis, rheumatoid arthritis, inflammatory bowel disease, transplant rejection and multiple sclerosis.

### DETAILED DESCRIPTION OF THE INVENTION

We have discovered that the position and type of the substituents on the phenyl group of the phenylalanine amino acid, in combination with stereochemistry, have a significant effect on the α₄β₁ and α₄β₇ integrin receptor antagonist activity of the aza-bridged-bicyclic compounds of the present invention. Relative to the above generic description, certain compounds having Formula (I) are preferred.

The preferred embodiments of the aza-bridged-bicyclic compounds of the present invention include those compounds wherein the phenyl group of the phenylalanine amino acid is substituted on the para position with R₆.

Preferred embodiments of the instant compounds also include those aza-bridged-bicyclic compounds wherein R₆ is benzofused heterocyclyl, aryl, arylamido, heteroarylamido, ureido (wherein the terminal amino is dialkyl substituted), aminocarbonyloxy (wherein amino is dialkyl substituted) and aryl(C₁₋₈)alkoxy. Experimental results seem to demonstrate that the activity of certain compounds as α₄β₁ and α₄β₇ integrin receptor antagonists increases significantly when the aryl and heteroaryl portion of R₆ is further mono- or di-substituted at the ortho position.

In addition to the above discoveries relative to the structure of the compounds of the present invention, we have experimentally determined that the stereochemistry significantly affects the α₄β₁ and α₄β₇ integrin receptor antagonist activity of certain compounds. In addition to racemic mixtures demonstrating activity as α₄β₁ and α₄β₇ integrin receptor antagonists, experimental results have shown that individual diastereomers each have either a significantly increased or significantly decreased activity as an α₄β₁ and α₄β₇ integrin receptor antagonist.

Although the racemic mixtures have significant activity compared to the resolved diastereomers, the (S,S) diastereomers appear to generally have higher activity than the (R,S) diastereomers. The scope of the present invention is intended to encompass all racemic mixtures, enantiomers and diastereomers including, but not limited to, (R/S,S), (R/S,R), (S,R/S), (R,R/S), (S,S), (R,S), (S,R) and (R,R) diastereomers and enantiomers of the compounds of the present invention without limitation.

Preferred embodiments are those aza-bridged-bicyclic compounds wherein Y is selected from the group consisting of hydrogen, C(O)(CH₂)₀₋₄R₁₈, —C(O)(CH₂)_{q}NC(O)R₁, —C(O)(CH₂)_{q}SR₁, —C(O)(CH₂)_{q}SOR₁; and —C(O)(CH₂)_{q}SO₂R₁wherein q is an integer from 1 to 4. More preferably, Y is selected from C(O)R₁₈, —C(O)(CH₂)_{q}NC(O)R₁, —C(O)(CH₂)_{q}SR₁, —C(O)(CH₂)_{q}SOR₁; and —C(O)(CH₂)_{q}SO₂R₁ wherein q is an integer from 1 to 2 Most preferably Y is —C(O)(CH₂)_{q}SR₁,

Preferred embodiments include those compounds wherein R₁ is selected from R₇. R₇ is preferably selected from the group consisting of aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃. Most preferred embodiments include those compounds wherein R₇ is selected from the group consisting of benzo-fused heterocyclyl (e.g. methylenedioxyphenyl), tolyl, phenyl and thienyl.

Preferred embodiments include those compounds wherein R₂, R₃, and R₅ are independently selected from the group consisting of hydrogen and C₁₋₄alkyl. More preferably, R₂, R₃, and R₅ are independently selected from the group consisting of hydrogen and methyl.

Preferred embodiments of the present invention include compounds wherein R₄ is selected from the group consisting of hydrogen and C₁₋₄alkyl. More preferably, R₄ is independently selected from the group consisting of hydrogen and methyl.

Preferred embodiments include those compounds wherein R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁)SO₂—R₁₀, —N(R₁₁)C(O)—N(R₁₁, R₁₂), —N(R₁₁)C(O)—N(R₁₂,R₁₇), —OC(O)—N(R₁₁,R₁₂), —OC(O)—N(R₁₂,R₁₇), —OC(O)—R₁₀ and R₁₀-(C₁₋₈)alkoxy. More preferably, R₆ is optionally present and is one to three substituents independently selected from the group consisting of halogen, C₁₋₄alkoxy, R₁₀, R₁₂, —N(R₁₁)C(O)—R₁₀, —N(R₁₁)C(O)—R₁₂, —N(R₁₁)SO₂—R₁₀, —N(R₁₁)C(O)—N(R₁₁,R₁₂), —N(R₁₁)C(O)—N(R₁₂,R₁₇), —OC(O)—N(R₁₁,R₁₂), —OC(O)—N(R₁₂,R₁₇), —OC(O)—R₁₀ and R₁₀-(C₁₋₄)alkoxy. Most preferably, R₆ is one substituent selected from the group consisting of R₁₀, —N(R₁₁)C(O)—R₁₀, —N(R₁₁)C(O)—N(R₁₁,R₁₂), —N(R₁₁)C(O)—N(R₁₂,R₁₇), —OC(O)—N(R₁₁,R₁₂), —OC(O)—N(R₁₂,R₁₇), —OC(O)—R₁₀ and R₁₀-methoxy. It is preferred that R₆ attachment is at the 4 positoin of the phenyl ring of the scaffold. Most preferably R₆ will be —N(R₁₁)C(O)—R₁₀ preferably wherein R₁₁ is hydrogen and R₁₀ is heteroaryl.

Preferably R₈ is selected from the group consisting of C₁₋₄alkyl, C₂₋₄alkenyl, C₂₋₄alkynyl, C₁₋₄alkoxy and (halo)₁₋₃(C₁₋₄)alkyl; wherein C₁₋₄alkyl, C₂₋₄alkenyl, C₂₋₄alkynyl and C₁₋₈alkoxy are optionally substituted with one to three substituents independently selected from R₁₄;

Preferred embodiments include those compounds wherein R₁₀, is selected from the group consisting of cycloalkyl, heterocyclyl, aryl and heteroaryl optionally substituted with one to five substituents independently selected from the group consisting of halogen, C₁₋₈alkyl, C₁₋₈alkoxy, C₁₋₈alkoxycarbonyl, carboxyl, arylcarbonyl, arylsulfonyl, —CF₃ and —OCF₃; wherein cycloalkyl and heterocyclyl are optionally substituted with one to three oxo substituents; and, wherein the aryl portion of the arylcarbonyl substituent is optionally substituted with one to five substituents independently selected from C₁₋₈alkoxy.

More preferably R₁₀ is selected from the group consisting of cycloalkyl, heterocyclyl, aryl and heteroaryl optionally substituted with one to five substituents independently selected from the group consisting of halogen, C₁₋₄alkyl, C₁₋₄alkoxy, C₁₋₄alkoxycarbonyl, carboxyl, arylcarbonyl, arylsulfonyl, —CF₃ and —OCF₃; wherein cycloalkyl and heterocyclyl are optionally substituted with one to three oxo substituents; and, wherein the aryl portion of the arylcarbonyl substituent is optionally substituted with one to five substituents independently selected from C₁₋₄alkoxy.

Most preferably R₁₀ is selected from the group consisting of cyclopropyl, 1,3-dihydro-2H-isoindolyl, 2-azabicyclo[2.2.2]octyl, piperidinyl, morpholinyl, phenyl, naphthalenyl, thienyl, 1H-pyrrolyl and pyridinyl; wherein cyclopropyl, piperidinyl, morpholinyl, phenyl, naphthalenyl, thienyl, 1H-pyrrolyl and pyridinyl are optionally substituted with one to four substituents independently selected from the group consisting of chlorine, fluorine, bromine, methyl, isopropyl, t-butyl, methoxy, t-butoxycarbonyl, carboxyl, phenylcarbonyl (wherein the phenyl portion of phenylcarbonyl is optionally substituted with one to two substituents selected from methoxy), —CF₃ and —OCF₃; wherein 1,3-dihydro-2H-isoindolyl is optionally substituted with oxo; and, wherein 2-azabicyclo[2.2.2]octyl is optionally substituted with phenylsulfonyl. In a preferred embodiment of the present invention R₁₀ is is 3,5 dichloropyridinyl and in a more preferred embodiment R₁₀ is attached at the 4 position thereof.

Preferred embodiments include those compounds wherein R₁₂ is selected from the group consisting of C₁₋₈alkyl and C₂₋₈alkynyl optionally substituted on a terminal carbon with R₁₄. More preferably, R₁₂ is selected from the group consisting of C₁₋₄alkyl and C₂₋₄alkynyl optionally substituted on a terminal carbon with R₁₄. Most preferably, R₁₂ is selected from the group consisting of t-butyl and ethynyl; wherein ethynyl is optionally substituted on a terminal carbon with a substituent R₁₄.

Preferred embodiments include those compounds wherein R₁₄ is preferably aryl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, arylcarbonyl, heteroarylcarbonyl, arylsulfonyl, amino, N-(C₁₋₈alkyl)amino, N,N-(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃. Most preferably R₁₄ is most preferably is selected from the group consisting of phenyl and C₁₋₈alkylphenyl.

Preferred embodiments include those compounds wherein R₁₁ is selected from the group consisting of hydrogen and C₁₋₄alkyl. More preferably, R₁₁ is hydrogen.

Preferred embodiments include those compounds wherein R₁₈ is selected from the group consisting of of hydroxy, C₁₋₄alkoxy, amino C₁₋₄alkyl, C₁₋₄alkyl amino C₁₋₄alkyl, diC₁₋₄alkylamino C₁₋₄alkyl, benzo-fused heterocyclyl, C₁₀polycycloalkyl and hydroxy C₁₋₄alkyl; wherein the benzo-fused heterocyclyl is substituted with C(O)R₁₉ and C(O)OR₁₉.

Preferred embodiments include those compounds wherein R₁₉ is selected from the group consisting of C₁₋₄alkyl, C₂₋₄alkenyl, C₂₋₄alkynyl, and (halo)₁₋₃(C₁₋₄)alkyl; wherein C₁₋₄alkyl, C₂₋₈alkenyl and C₂₋₄alkynyl are optionally substituted on a terminal carbon with one to three substituents independently selected from R₁₄;

Preferred embodiments include those compounds wherein A is selected from the group consisting of methylene and ethylene. Most preferably A is methylene.

Preferred embodiments include those compounds wherein B₁ and B₂ are independently selected from the group consisting of C₁₋₂alkylene and C₂alkenylene optionally substituted with one to two substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃.

More preferably, B₁ and B₂ are independently selected from the group consisting of —CH₂—, —(CH₂)₂— and —(CH)₂— optionally substituted with one to two substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃. Also more preferably, B₁ is selected from the group consisting of —CH₂—, —(CH₂)₂— and —(CH)₂— optionally substituted with one to two substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃ and that B₂ is selected from —(CH₂)₂—. Most preferably, B₁ is selected from the group consisting of —CH₂—, —(CH₂)₂— and —(CH)₂—.

Embodiments of the aza-bridged-bicyclic amino acid compounds of the present invention include those compounds of Formula (III) shown in Table I of the formula:

TABLE I

| | |
|---|---|
| | Formula III |
| 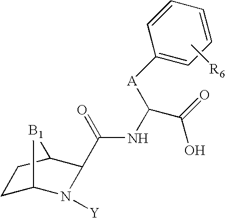 | |

Wherein Y, B₁, R₁, A and R₆ are as previously
defined and may be selected from the group consisting of:

| Cpd | B₁ | Y | R₁ | A | R₆ |
|---|---|---|---|---|---|
| 1 | (CH₂)₂ | C(O)(CH₂)S R₁ | 2-pyridinyl | CH₂ | 4-NHC(O)-(3,5-Cl₂)pyridin-4-yl |
| 2 | (CH₂)₂ | C(O)(CH₂)S R₁ | -4-Fphenyl | CH₂ | 4-NHC(O)-(3,5-Cl₂)pyridin-4-yl |
| 3 | (CH₂)₂ | C(O)(CH₂)S R₁ | 4-pyridinyl | CH₂ | 4-NHC(O)-(3,5-Cl₂)pyridin-4-yl |
| 4 | (CH₂)₂ | C(O)(CH₂)S R₁ | 2-pyridinyl | CH₂ | 4-OCH₂-(2,6-phenyl) |
| 5 | (CH₂)₂ | C(O)(CH₂)SO₂ R₁ | —CH₃ | CH₂ | 4-NHC(O)-(3,5-Cl₂)pyridin-4-yl |
| 6 | (CH₂)₂ | C(O)(CH₂)SO₂ R₁ | -phenyl | CH₂ | 4-NHC(O)-(3,5-Cl₂)pyridin-4-yl |
| 7 | (CH₂)₂ | C(O)(CH₂)₂NC(O) R₁ | —OC(CH₃)₃ | CH₂ | 4-NHC(O)-(3,5-Cl₂)pyridin-4-yl |

and pharmaceutically acceptable salts, racemic mixtures, diastereomers and salts thereof.

Embodiments of the aza-bridged-bicyclic amino acid compounds of the present invention include those compounds of Formula (IV) shown in Table II of the formula:

TABLE II

| | |
|---|---|
| | Formula IV |
| 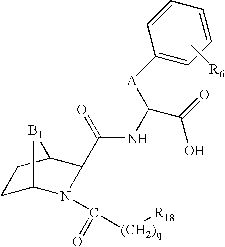 | |

Wherein B₁, R₁₈, q, A and R₆ are as previously
defined and more preferably are selected from the group consisting of:

| Cpd | B₁ | q | R₁₈ | A | R₆ |
|---|---|---|---|---|---|
| 8 | (CH₂)₂ | 2 | —OH | CH₂ | 4-NHC(O)-(3,5-Cl₂)pyridin-4-yl |
| 9 | (CH₂)₂ | 2 | —OH | CH₂ | 4-NHC(O)-(2,6-Cl₂2)phenyl |
| 10 | (CH₂)₂ | 2 | —OH | CH₂ | 4-OC(O)-morpholin-1-yl |
| 11 | (CH₂)₂ | 2 | CH₂C(CH₃)₂OH | CH₂ | 4-NHC(O)-(3,5-Cl₂)pyridin-4-yl |
| 12 | (CH₂)₂ | 2 | —OCH₃ | CH₂ | 4-NHC(O)-(3,5-Cl₂)pyridin-4-yl |
| 13 | (CH₂)₂ | 2 | —OCH₃ | CH₂ | 4-NHC(O)(CH₂)₂OCH₃ |
| 14 | (CH₂)₂ | 2 | —OCH₃ | CH₂ | 4-OC(O)N(CH₃)₂ |
| 15 | (CH₂)₂ | 2 | —NH₂ | CH₂ | 4-NHC(O)-(3,5-Cl₂)pyridin-4-yl |
| 16 | (CH₂)₂ | 2 | N(CH₃)₂ | CH₂ | 4-NHC(O)-(3,5-Cl₂)pyridin-4-yl |
| 17 | (CH₂)₂ | 2 | Adamantyl | CH₂ | 4-NHC(O)-(3,5-Cl₂)pyridin-4-yl |
| 18 | (CH₂)₂ | 1 | 2,3,4,5-tetrahydro-benzo[f]- | CH₂ | 4-NHC(O)-(3,5-Cl₂)pyridin-4-yl |
| | | | [1,4]oxazepine | | |
| 19 | (CH₂)₂ | 1 | 3,4,4a,8a-tetrahydro-1H- | | 4-NHC(O)-(3,5-Cl₂)pyridin-4-yl |
| | | | isoquinoline | | |
| 20 | (CH₂)₂ | 1 | 3,4,4a,8a-tetrahydro-1H- | CH₂ | 4-NHC(O)-(3,5-Cl₂)pyridin-4-yl |
| | | | isoquinoline-2-tert- | | |
| | | | butoxycarbonyl | | |
| 21 | (CH₂)₂ | 1 | 5-benzyl[1,3]dioxole | CH₂ | 4-NHC(O)-(3,5-Cl₂)pyridin-4-yl |
| 22 | (CH₂)₂ | 2 | 5-benzyl[1,3]dioxole | CH₂ | 4-NHC(O)-(2,6-Cl₂)phenyl |
| 23 | (CH₂)₂ | 2 | 5-benzyl[1,3]dioxole | CH₂ | 4-OC(O)-morpholin-1-yl |

and pharmaceutically acceptable salts, racemic mixtures, diastereomers and salts thereof.

The compounds of the present invention may also be present in the form of pharmaceutically acceptable salts. For use in medicine, the salts of the compounds of this invention refer to non-toxic “pharmaceutically acceptable salts” (Ref. International J. Pharm., 1986, 33, 201-217; J. Pharm. Sci., 1997 (January), 66, 1, 1). Other salts may, however, be useful in the preparations of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. Where the processes for the preparation of the compounds according to the invention give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diasteromers by standard techniques, such as the formation of stereoisomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers and enantiomers thereof are encompassed within the scope of the present invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Grouls in Organic Synthesis, John Wiley & Sons, 1991. The protecting groups may be removed at a convenient subsequent stage using methods known in the art.

Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

As used herein, unless otherwise noted, “alkyl” and “alkoxy” whether used alone or as part of a substituent group refers to straight and branched carbon chains having 1 to 8 carbon atoms or any number within this range. Similarly, alkenyl and alkynyl groups include straight and branched chain alkenes and alkynes having 2 to 8 carbon atoms or any number within this range, wherein an alkenyl chain has at least one double bond in the chain and an alkynyl chain has at least one triple bond in the chain. Alkoxy radicals are oxygen ethers formed from the previously described straight or branched chain alkyl groups.

As used herein, unless otherwise noted “oxo” whether used alone or as part of a substituent group refers to an O═ to either a carbon or a sulfur atom. For example, phthalimide and saccharin are examples of compounds with oxo substituents.

The term “cycloalkyl,” as used herein, refers to an optionally substituted, stable, saturated or partially saturated monocyclic or bicyclic ring system containing from 3 to 8 ring carbons and preferably 5 to 7 ring carbons. Examples of such cyclic alkyl rings include cyclopropyl, cyclobutyl, cyclopentyl, cyclohexyl or cycloheptyl.

The term “polycycloalkyl” as used herein, refers to an optionally substituted, stable, saturated or partially saturated multicyclic ring system contain from 5 to 10 with at least one carbon bridge between the rings. Examples of such polycyclic alkyl rings include bicyclo[1.1.1]pentanyl, bicyclo[2.1.1]hexanyl, bicyclo[2.2.1]heptanyl, bicyclo[2.2.2.]octanyl, bicyclo[3.3.0]octanyl, bicyclo[3.2.1]octanyl and adamatanyl.

The term benzo-fused cycloalkyl” shall mean a bicyclic ring structure wherein one of the rings is a phenyl and the other is a five to six membered cycolalkyl. Examples of such benzo-fused cycloalkyl include but are not limited to indanyl, fluorenyl and the like.

The term “heterocyclyl” as used herein refers to an optionally substituted, stable, saturated or partially saturated 5 or 6 membered monocyclic or bicyclic ring systems which consists of carbon atoms and from one to three heteroatoms selected from N, O or S. Examples of heterocyclyl groups include, but are not limited to, pyrrolinyl (including 2H-pyrrole, 2-pyrrolinyl or 3-pyrrolinyl), pyrrolidinyl, dioxolanyl, 2-imidazolinyl, imidazolidinyl, 2-pyrazolinyl, pyrazolidinyl, piperidinyl, dioxanyl, morpholinyl, dithianyl, thiomorpholinyl or piperazinyl. The heterocyclyl group may be attached at any heteroatom or carbon atom, which results in the creation of a stable structure.

The term “benzo-fused heterocyclyl as used herein, refers to a heterocycle ring that is fused with a phenyl ring to form a multiple ring system. Examples of which include compounds selected from the group consisting of benzyl[1,3]dioxole, 2,3,4,5-tetrahydrobenzo[f]-[1,4]oxazepinyl; 3,4,4a,8a-tetrahydro-1H-isoquinolinyl and the like.

The term “aryl”, as used herein, refers to optionally substituted aromatic groups comprising a stable six membered monocyclic or ten membered bicyclic aromatic ring system, which consists of carbon atoms. Examples of aryl groups include, but are not limited to, phenyl or naphthalenyl.

The term “heteroaryl” as used herein represents a stable five or six membered monocyclic aromatic ring system or a nine or ten membered benzo-fused heteroaromatic ring system which consists of carbon atoms and from one to three heteroatoms selected from N, O or S. The heteroaryl group may be attached at any heteroatom or carbon atom which results in the creation of a stable structure.

The term “arylalkyl” means an alkyl group substituted with an aryl group (e.g., benzyl, phenethyl). The term “arylalkoxy” indicates an alkoxy group substituted with an aryl group (e.g., benzyloxy, phenethoxy, etc.). Similarly, the term “aryloxy” indicates an oxy group substituted with an aryl group (e.g., phenoxy).

Whenever the term “alkyl” or “aryl” or either of their prefix roots appear in a name of a substituent (e.g., aralkyl, alkylamino) it shall be interpreted as including those limitations given above for “alkyl” and “aryl.” Designated numbers of carbon atoms (e.g., C₁₋₆) shall refer independently to the number of carbon atoms in an alkyl or cycloalkyl moiety or to the alkyl portion of a larger substituent in which alkyl appears as its prefix root.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The aza-bridged-bicyclic amino acid compounds of the present invention are useful α4 integrin receptor antagonists and, more particularly, α4β1 and α4β7 integrin receptor antagonists for treating a variety of integrin mediated disorders that are ameliorated by inhibition of the α4β1 and α4β7 integrin receptor including, but not limited to, inflammatory, autoimmune and cell-proliferative disorders.

Illustrative of the invention is a pharmaceutical composition comprising a pharmaceutically acceptable carrier and any of the compounds described above. Also illustrative of the invention is a pharmaceutical composition made by mixing any of the compounds described above and a pharmaceutically acceptable carrier. A further illustration of the invention is a process for making a pharmaceutical composition comprising mixing any of the compounds described above and a pharmaceutically acceptable carrier. The present invention also provides pharmaceutical compositions comprising one or more compounds of this invention in association with a pharmaceutically acceptable carrier.

An example of the invention is a method for the treatment of integrin mediated disorders in a subject in need thereof comprising administering to the subject a therapeutically effective amount of any of the compounds or pharmaceutical compositions described above. Also included in the invention is the use of a compound of Formula (I) for the preparation of a medicament for treating an integrin mediated disorder in a subject in need thereof.

Further exemplifying the invention is the method for the treatment of integrin mediated disorders, wherein the therapeutically effective amount of the compound is from about 0.01 mg/kg/day to about 30 mg/kg/day.

In accordance with the methods of the present invention, the individual components of the pharmaceutical compositions described herein can be administered separately at different times during the course of therapy or concurrently in divided or single combination forms. The instant invention is therefore to be understood as embracing all such regimes of simultaneous or alternating treatment and the term “administering” is to be interpreted accordingly.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, that is being sought by a researcher, veterinarian, medical doctor, or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The utility of the compounds to treat integrin mediated disorders can be determined according to the procedures herein. The present invention therefore provides a method for the treatment of integrin mediated disorders in a subject in need thereof which comprises administering any of the compounds as defined herein in a quantity effective to inhibit the α4β1 and α4β7 integrin receptor including, but not limited to, inflammatory, autoimmune and cell-proliferative disorders. Accordingly, a compound of the present invention may be administered by any conventional route of administration including, but not limited to oral, nasal, pulmonary, sublingual, ocular, transdermal, rectal, vaginal and parenteral (i.e. subcutaneous, intramuscular, intradermal, intravenous etc.).

To prepare the pharmaceutical compositions of this invention, one or more compounds of Formula (I) or salt thereof as the active ingredient, is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing a pharmaceutical composition of the present invention in liquid dosage form for oral, topical and parenteral administration, any of the usual pharmaceutical media or excipients may be employed. Thus, for liquid dosage forms, such as suspensions (i.e. colloids, emulsions and dispersions) and solutions, suitable carriers and additives include but are not limited to pharmaceutically acceptable wetting agents, dispersants, flocculation agents, thickeners, pH control agents (i.e. buffers), osmotic agents, coloring agents, flavors, fragrances, preservatives (i.e. to control microbial growth, etc.) and a liquid vehicle may be employed. Not all of the components listed above will be required for each liquid dosage form.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg/kg to about 300 mg/kg (preferably from about 0.01 mg/kg to about 100 mg/kg; and, more preferably, from about 0.01 mg/kg to about 30 mg/kg) and may be given at a dosage of from about 0.01 mg/kg/day to about 300 mg/kg/day (preferably from about 0.01 mg/kg/day to about 100 mg/kg/day and more preferably from about 0.01 mg/kg/day to about 30 mg/kg/day). Preferably, the method for the treatment of integrin mediated disorders described in the present invention using any of the compounds as defined herein, the dosage form will contain a pharmaceutically acceptable carrier containing between from about 0.01 mg to about 100 mg; and, more preferably, from about 5 mg to about 50 mg of the compound, and may be constituted into any form suitable for the mode of administration selected. The dosages, however, may be varied depending upon the requirement of the subjects, the severity of the condition being treated and the compound being employed. The use of either daily administration or post-periodic dosing may be employed.

Preferably these compositions are in unit dosage forms from such as tablets, pills, capsules, dry powders for reconstitution or inhalation, granules, lozenges, sterile parenteral solutions or suspensions, metered aerosol or liquid sprays, drops, ampoules, autoinjector devices or suppositories for administration by oral, intranasal, sublingual, intraocular, transdermal, parenteral, rectal, vaginal, dry powder inhaler or other inhalation or insufflation means. Alternatively, the composition may be presented in a form suitable for once-weekly or once-monthly administration; for example, an insoluble salt of the active compound, such as the decanoate salt, may be adapted to provide a depot preparation for intramuscular injection.

For preparing solid pharmaceutical compositions such as tablets, the principal active ingredient is mixed with a pharmaceutical carrier, e.g. conventional tableting ingredients such as diluents, binders, adhesives, disintegrants, lubricants, antiadherents and gildants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol-sorbitol, microcrystalline cellulose (i.e. AVICEL™ microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE™ available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL™ silica available from Cabot, SYLOID™ silica available from W.R. Grace/Davison, and AEROSIL™ silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Generally these carriers are mixed with the pharmaceutical active to form a solid preformulation composition containing a homogeneous mixture of the pharmaceutical active of the present invention, or a pharmaceutically acceptable salt thereof. Generally the preformulation will be formed by one of three common methods: (a) wet granulation, (b) dry granulation and (c) dry blending. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.1 mg to about 500 mg of the active ingredient of the present invention. The tablets or pills containing the novel compositions may also be formulated in multilayer tablets or pills to provide a sustained or provide dual-release products. For example, a dual release tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric materials such as shellac, cellulose acetate (i.e. cellulose acetate phthalate, cellulose acetate trimetilitate), polyvinyl acetate phthalate, hydroxypropyl methylcellulose phthalate, hydroxypropyl methylcellulose acetate succinate, methacrylate and ethylacrylate copolymers, methacrylate and methyl methacrylate copolymers and the like. Sustained release tablets may also be made by film coating or wet granulation using slightly soluble or insoluble substances in solution (which for a wet granulation acts as the binding agents) or low melting solids a molten form (which in a wet granulation may incorporate the active ingredient). These materials include natural and synthetic polymers waxes, hydrogenated oils, fatty acids and alcohols (i.e. beeswax, carnauba wax, cetyl alcohol, cetylstearyl alcohol, and the like), esters of fatty acids metallic soaps, and other acceptable materials that can be used to granulate, coat, entrap or otherwise limit the solubility of an active ingredient to achieve a prolonged or sustained release product.

The liquid forms in which the novel compositions of the present invention may be incorporated for administration orally or by injection include, but are not limited to aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable suspending agents for aqueous suspensions, include synthetic and natural gums such as, acacia, agar, alginate (i.e. propylene alginate, sodium alginate and the like), guar, karaya, locust bean, pectin, tragacanth, and xanthan gum, cellulosics such as sodium carboxymethylcellulose, methylcellulose, hydroxymethylcellulose, hydroxyethylcellulose, hydroxypropyl cellulose and hydroxypropyl methylcellulose, and combinations thereof, synthetic polymers such as polyvinyl pyrrolidone, carbomer (i.e. carboxypolymethylene), and polyethylene glycol; clays such as bentonite, hectorite, attapulgite or sepiolite; and other pharmaceutically acceptable suspending agents such as lecithin, gelatin or the like. Suitable surfactants include but are not limited to sodium docusate, sodium lauryl sulfate, polysorbate, octoxynol-9, nonoxynol-10, polysorbate 20, polysorbate 40, polysorbate 60, polysorbate 80, polyoxamer 188, polyoxamer 235 and combinations thereof. Suitable deflocculating or dispersing agent include pharmaceutical grade lecithins. Suitable flocculating agent include but are not limited to simple neutral electrolytes (i.e. sodium chloride, potassium, chloride, and the like), highly charged insoluble polymers and polyelectrolyte species, water soluble divalent or trivalent ions (i.e. calcium salts, alums or sulfates, citrates and phosphates (which can be used jointly in formulations as pH buffers and flocculating agents). Suitable preservatives include but are not limited to parabens (i.e. methyl, ethyl, n-propyl and n-butyl), sorbic acid, thimerosal, quaternary ammonium salts, benzyl alcohol, benzoic acid, chlorhexidine gluconate, phenylethanol and the like. There are many liquid vehicles that may be used in liquid pharmaceutical dosage forms, however, the liquid vehicle that is used in a particular dosage form must be compatible with the suspending agent(s). For example, nonpolar liquid vehicles such as fatty esters and oils liquid vehicles are best used with suspending agents such as low HLB (Hydrophile-Lipophile Balance) surfactants, stearalkonium hectorite, water insoluble resins, water insoluble film forming polymers and the like. Conversely, polar liquids such as water, alcohols, polyols and glycols are best used with suspending agents such as higher HLB surfactants, clays silicates, gums, water soluble cellulosics, water soluble polymers and the like. For parenteral administration, sterile suspensions and solutions are desired. Liquid forms useful for parenteral administration include sterile solutions, emulsions and suspensions. Isotonic preparations which generally contain suitable preservatives are employed when intravenous administration is desired.

Furthermore, compounds of the present invention can be administered in an intranasal dosage form via topical use of suitable intranasal vehicles or via transdermal skin patches, the composition of which are well known to those of ordinary skill in that art. To be administered in the form of a transdermal delivery system, the administration of a therapeutic dose will, of course, be continuous rather than intermittent throughout the dosage regimen.

Compounds of the present invention can also be administered in the form of liposome delivery systems, such as small unilamellar vesicles, large unilamellar vesicles, multilamellar vesicles and the like. Liposomes can be formed from a variety of phospholipids, such as cholesterol, stearylamine, phosphatidylcholines and the like.

Compounds of the present invention may also be delivered by the use of monoclonal antibodies as individual carriers to which the compound molecules are coupled. The compounds of the present invention may also be coupled with soluble polymers as targetable drug carriers. Such polymers can include, but are not limited to polyvinylpyrrolidone, pyran copolymer, polyhydroxypropylmethacrylamidephenol, polyhydroxy-ethylaspartamidephenol, or polyethyl eneoxidepolylysine substituted with palmitoyl residue. Furthermore, the compounds of the present invention may be coupled to a class of biodegradable polymers useful in achieving controlled release of a drug, for example, to homopolymers and copolymers (which means polymers containing two or more chemically distinguishable repeating units) of lactide (which includes lactic acid d-, l- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone,hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, polyorthoesters, polyacetals, polydihydropyrans, polycyanoacrylates and cross-linked or amphipathic block copolymers of hydrogels and blends thereof.

Compounds of this invention may be administered in any of the foregoing compositions and dosage regimens or by means of those compositions and dosage regimens established in the art whenever treatment of integrin mediated disorders is required for a subject in need thereof.

The daily dose of a pharmaceutical composition of the present invention may be varied over a wide range from about 0.7 mg to about 21,000 mg per adult human per day; preferably, the dose will be in the range of from about 0.7 mg to about 7000 mg per adult human per day; most preferably the dose will be in the range of from about 0.7 mg to about 2100 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated. An effective amount of the drug is ordinarily supplied at a dosage level of from about 0.01 mg/kg to about 300 mg/kg of body weight per day. Preferably, the range is from about 0.01 mg/kg to about 100 mg/kg of body weight per day; and, most preferably, from about 0.01 mg/kg to about 30 mg/kg of body weight per day. Advantageously, a compound of the present invention may be administered in a single daily dose or the total daily dosage may be administered in divided doses of two, three or four times daily.

Optimal dosages to be administered may be readily determined by those skilled in the art, and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level.

Abbreviations used in the instant specification, particularly the Schemes and Examples, are as follows:
BSA Bovine Serum Albumen
DBC 2,6-Dichlorobenzoylchloride
DCM Dichloromethane
DIEA Diisopropylethylamine
DMAP Dimethylaminopyridine
DMF N,N-Dimethylformamide
EDAC N-ethyl-N′-dimethylaminopropylcarbodiimide hydrochloride
Et₂O Diethyl ether
EtOAc Ethyl acetate
EtOH Ethanol
h hour
HOBt Hydroxybenzotriazole hydrate
HATU O-(7-azabenzotriazol-1-yl)-1,1,3,3-tetramethyluronium hexafluorophosphate
HPLC High Performance Liquid Chromatography
NMM N-methyl-morpholine
Me Methyl
MeOH Methanol
min Minutes
PBS Phosphate Buffer Solution
Ph Phenyl
rt Room temperature
SDS Sodium Dodecasulfate
TEA Triethylamine
THF Tetrahydrofuran
Thi Thienyl
TMS Tetramethylsilane
TFA Trifluoroacetic acid
Tol Toluene

### General Synthetic Methods

Representative compounds of the present invention can be synthesized in accordance with the general synthetic methods described below and are illustrated more particularly in the schemes that follow. Since the schemes are an illustration, the invention should not be construed as being limited by the chemical reactions and conditions expressed. The preparation of the various starting materials used in the schemes is well within the skill of persons versed in the art.

In the following general method for preparing compounds of the invention, amino ester Compound A1 is protected with a conventional amino protecting group to give Compound A2, which is saponified under basic conditions to yield carboxylic acid Compound A3. Compound A3 is condensed with Compound A4 in the presence of an appropriate coupling agent, base, and solvent. An appropriate coupling agent may include, and is not limited to, EDAC hydrochloride, DIC, EDC, DCC or HATU; an appropriate base may include but is not limited to, DIEA; and an appropriate solvent may include, but is not limited to, DCM or DMF. For compounds of the present invention, (S)-4-Nitrophenylalanine methyl ester is acylated with Compound A3 in the presence of EDC, HOBt, and DIEA in DCM.

The nitro group of Compound A5 is reduced with zinc powder to afford the corresponding amine, Compound A6. The amine of Compound A6 is acylated with a variety of acid chlorides to provide a variety of amides, represented by Compound A7. For example R_{A} may be selected from the group consisting of —R₁₀, —R₁₂, —N(R₁₁,R₁₀), —N(R₁₁,R₁₂), and —N(R₁₂,R₁₇). Sulfamides could be made by analogues procedures. Compound A7 is deprotected under acidic conditions to provide the resultant amino Compound A8 which is then acylated by several methods: Compound A8 may be condensed with carboxylic acids in the presence of an appropriate coupling agent, base, and solvent. For example, R_{B} may be selected from the group consisting of —(CH₂)₀₋₄R₁₈, —(CH₂)_{q}NC(O)R₁, —(CH₂)_{q}SR₁, —(CH₂)_{q}SOR₁; and —(CH₂)_{q}SO₂R₁. Compounds of the present invention were made in the presence of HOBt, EDC, NMM in DCM; similarly, Compound A8 may be condensed with an appropriate acid chloride to provide Compound A9. The ester of Compound A9 is saponified under basic conditions to yield Compound A10.

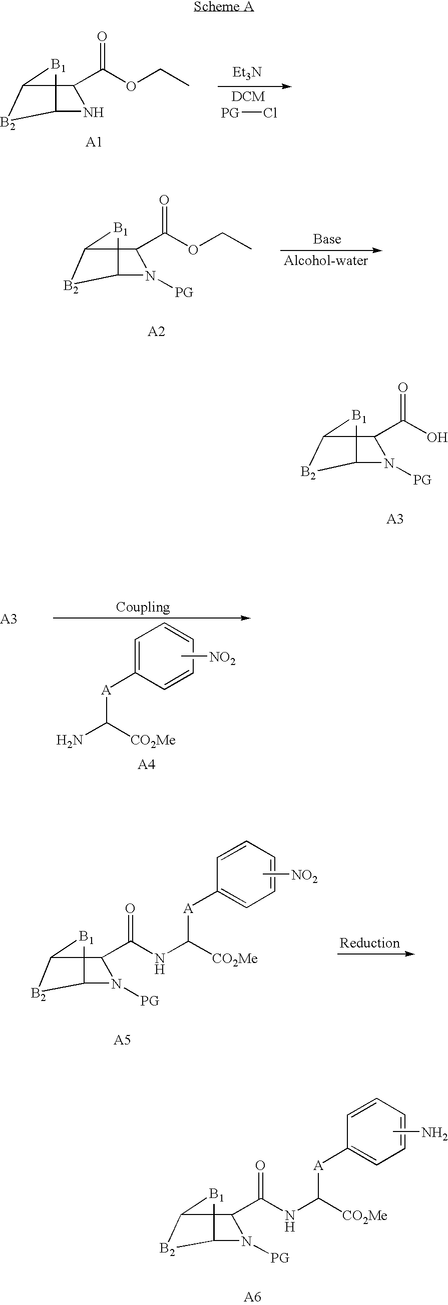

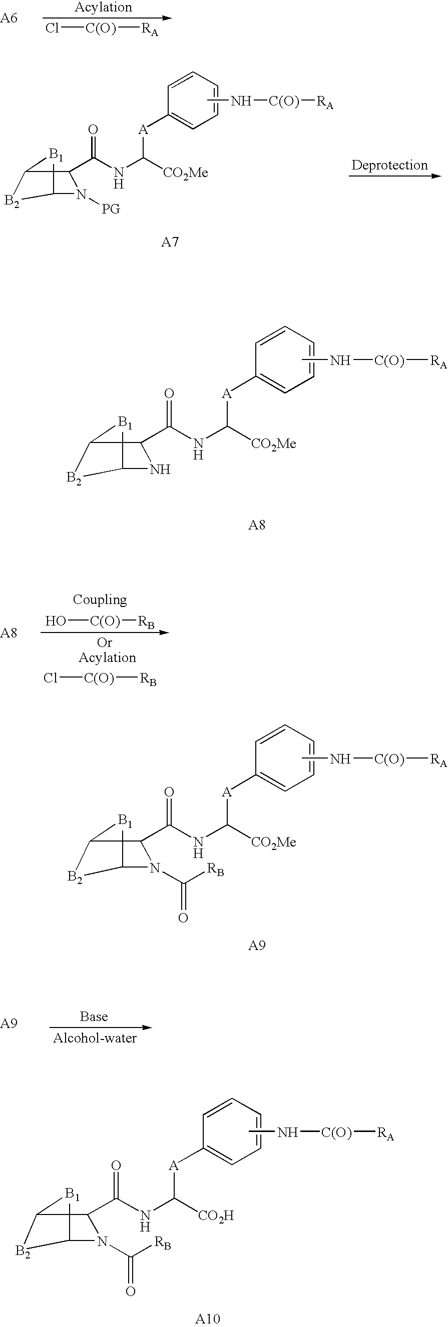

Scheme B describes the preparation of compounds of the present invention in which substituents of the phenyl group are attached through an oxygen atom. Compound A3 is coupled with Compound B1 in the presence of an appropriate coupling agent, base, and solvent, as described in Scheme A. If B1 is to be substituted with only —O—R₁₀ or R₁₀—C₁₋₈ alkoxy B1 the appropriate substituted phenyl group may be used as a starting material and the acylation step skipped.

The hydroxyl group of Compound B2 is acylated with acid chloride. The R_{C} group is an acylated group listed in R₆ selected from the group consisting of —R₁₀, —R₁₂, —N(R₁₁,R₁₀), —N(R₁₁,R₁₂), and —N(R₁₂,R₁₇). R_{B} is as defined in Scheme A. Other compounds of the present invention having a variety of substituents attached to the oxygen atom may be made by reacting Compound B2 with a variety of acid chlorides.

Compound B3 is deprotected using hydrogenation to provide the resultant amino Compound B4 which is acylated by several methods: Compound B4 may be condensed with carboxylic acids in the presence of an appropriate coupling agent, base, and solvent. Compounds of the present invention were made in the presence of HOBt, EDC, NMM in DCM; similarly, Compound B4 may be condensed with an appropriate acid chloride to provide Compound B5. The ester of Compound B5 is saponified under basic conditions to yield Compound B6.

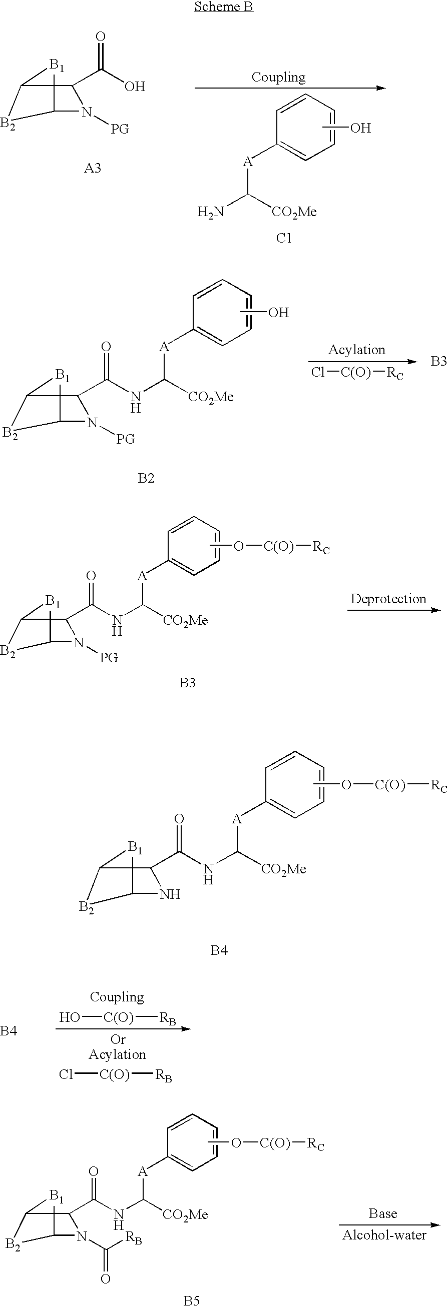

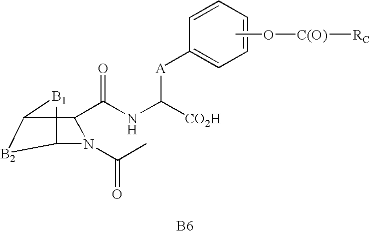

### EXAMPLE 1

Amino ester Compound 1a (5.90 g, 0.0322 mol) was dissolved in dry DCM (100 mL) containing TEA (9.43 mL, 0.067 mol) and the solution was cooled in an ice bath. Benzyl chloroformate Compound 1b (4.83 mL, 0.0338 mol) was added dropwise over a 45 min period. The reaction was stirred for 2 h at 0° C., after which time the reaction was warmed to rt and stirred an additional 18h. The reaction mixture was washed with 0.1 N HCl, 5% NaHCO₃, and water before being dried (MgSO₄) and concentrated to a viscous oil. The product was analyzed by TLC (hexane:EtOAc 1:1, R_{f} 0.75). The crude material was purified by column chromatography (silica gel, hexane:EtOAc, 7:1) to give 7.73 g (76%) of Compound 1c as a viscous oil. ¹H NMR (300 MHz, CDCl₃) δ 7.37-7.26 (5H, m), 5.20-5.10 (2H, m), 4.71-4.69 (1H, m), 4.26-4.00 (3H, m), 2.24-2.22 (1H, m), 2.16-2.15 and 2.13-2.04 (1H, m), 2.00-1.40 (m, 9H), 1.25 and 1.15 (3H, J=7.3 Hz).

Compound 1c (7.73 g, 24.4 mmol) was dissolved in MeOH (200 mL) and 1.0 N KOH (122 mL, 122 mmol) was added as one portion. The reaction was warmed to 70° C. and stirred for 10 h, and the MeOH was evaporated. The residue was dissolved in water (100 mL), acidified with 1N HCl to pH 2, and extracted with EtOAc (3×100 mL). The organic fractions were combined, dried (MgSO₄), filtered, and evaporated to provide Compound 1d as a white solid (6.34 g, 90%). ¹H NMR (300 MHz, CDCl₃) δ 7.34-7.26 (5H, m), 5.29-5.10 (2H, m), 4.70-4.13 (2H, m), 2.29-2.23 (1H, m), 2.09-2.00 (1H, m), 2.00-1.40 (8H, m); MS(ES⁻) 288.

Compound 1d (2.89 g, 10.0 mmol) was dissolved in dry DCM (50 mL) containing EDC (2.11 g, 11 mmol) HOBt (1.42 g, 11 mmol) and DIEA (2.42 mL, 24.2 mmol). Compound 1e (2.60 g, 11 mmol) was added as one portion, and reaction was stirred under N₂ for 3 h at rt. The reaction mixture was washed with water (100 mL), 10% citric acid solution, and 5% NaHCO₃ aqueous solution, then dried (MgSO₄), filtered, and evaporated. The residue (yellow foam, 4.58 g) was purified by column chromatography (silica gel, hexane:EtOAc 1:1; R_{f} 0.59) providing 3.78 g (76%) of Compound 1f: ¹H NMR (300 MHz, CDCl₃) δ 8.13 and 8.11 (2H, s), 7.40-7.00 (7H, m), 6.80-6.60 (1H, broad s), 5.17-5.12 (2H, broad s), 4.95-4.89 (1H, m), 4.14-4.00 (1H, m), 3.72 and 3.59 (3H, s), 3.35-3.05 (2H, m), 2.10-2.02 (1H, m), 2.00-1.20 (8H, m); MS(ES⁺) 496.

A solution of Compound 1f (3.54 g, 7.14 mmol) in MeOH (70 mL) was placed in the round-bottom flask equipped with mechanical stirrer and reflux condenser, containing Zn powder (4.67 g, 71.4 mmol) and NH₄Cl (1.91 g, 35.7 mmol). The mixture was stirred for 3 h at 65° C. then cooled to rt and filtered through Celite. The clear solution was concentrated to approximately 20 mL, then diluted with 10% NaHCO₃ (200 mL) and extracted with EtOAc (4×20 mL). The organic fractions were combined, dried (MgSO₄), filtered and evaporated. The residue was purified by column chromatography (silica gel, EtOAc:hexane 1:1, R_{f} 0.11) resulting in 3.19 g (96%) Compound 1g: ¹H NMR (300 MHz, CDCl₃) δ 7.35-7.10 (7H, m), 6.90-6.75 (2H, m), 6.57 (2H, d, J=8.3 Hz), 6.55 (1H, m), 5.15-5.05 (2H, m), 4.85-4.81 (1H, m), 4.15-4.08 (2H, m), 3.69-3.59 (3H, m), 3.00-2.93 (2H, m), 2.1-2.00 (1H, m), 1.90-1.20 (8H, m); MS(ES⁺) 466.

The Compound 1h (1.50 g, 7.81 mmol) was placed in a round-bottom flask equipped with a stirrer and reflux condenser. DCM (20 mL) containing 2 drops of DMF was added as one portion followed by thionyl chloride (0.85 mL, 11.7 mmol). The reaction mixture was refluxed for 3 h resulting in a clear solution. The solution was evaporated under vacuum to yield a yellow oil, Compound 1i, which was used in the next step without purification.

Compound 1g (2.88 g, 6.19 mmol) was dissolved in DCM (50 mL) containing TEA (2.23 mL, 16 mmol) and placed in a round-bottom flask equipped with a mechanical stirrer and immersed in an ice bath. A solution of Compound 1 i in DCM (20 mL) was added dropwise over 45 min and the reaction was allowed to warm to rt while stirring overnight. The solution was washed with 10% NaHCO3, 0.1 N HCl, and water, then dried (MgSO₄), filtered, and evaporated. The residue was purified by column chromatography (silica gel, EtOAc) to give 3.65 g (92%) of Compound 1j: ¹H NMR (300 MHz, CD₃CN) δ 8.90 (1H, s), 8.65 (2H, s), 7.65-7.50 (2H, m), 7.45-7.15 (9H, m), 6.97 (1H, d, J=8.0 Hz), 5.12-4.99 (2H, m), 4.75-4.60 (1H, m), 4.09-4.01 (2H, m), 3.69-3.62 (3H, m), 3.20-2.90 (1H, m); MS(ES⁺) 639.

Compound 1j (3.65 g, 5.72 mmol) was added to 33% HBr in AcOH (45 mL) under vigorous stirring. The reaction was kept at rt for 3 h (reaction became homogeneous after 45 min). The viscous liquid was evaporated under vacuum, and the resulting residue was dissolved in water (250 mL), then extracted with Et₂O. The organic layer was discarded. The aqueous layer was basified to pH 7 with Na₂CO₃ and extracted with EtOAc (5×20 mL). The organic layers were combined, dried (Na₂SO₄) and evaporated to provide 2.85 g of a pale yellow solid, Compound 1 k. Compound 1 k was purified by column chromatography (CHCl₃:MeOH, 9:1) to give 2.5 g (87%) of pure Compound 1k as a white solid: ¹H NMR (300 MHz, DMSO-d₆) δ 8.80 (2H, s), 8.48 (1H, d, J=8.3 Hz), 7.57 (2H, d, J=8.5 Hz), 7.21 (2H, d, J=8.5 Hz), 4.69-4.61 (1H, m), 3.66 (3H, s), 3.13-3.00 (2H, m), 2.76 (2H, m), 2.50 (1H, broad s), 1.84 (1H, broad s), 1.62-1.08 (9H, m); MS(ES⁺) 505.

Compound 1k (51 mg, 0.10 mmol), 3-BOC-amino propanoic acid Compound 1l (16 mg, 0.105 mmol), EDC hydrochloride (21 mg, 0.11 mmol), and HOBt (14 mg, 0.105 mmol) were suspended in DCM (1 mL) at rt and N-methyl-morpholine (14 μL, 0.120 mmol) was added in one portion. The reaction was kept at rt for 4 h and loaded into a silica column. Flash chromatography (silica gel, EtOAc) provided 40 mg (59%) of Compound 1m as a white solid: MS(ES⁺) 676.

Compound 1m (40 mg, 0.06 mmol) was dissolved in MeOH:water (2 mL, 1:1) and LiOH (4 mg, 0.1 mmol) was added in one portion. The reaction was homogenized in an ultrasonic bath and kept overnight at rt. The reaction mixture was diluted with water (20 mL), extracted with Et₂O (10 mL), and the organic layer was discarded. The aqueous layer was acidified with 1 N HCl to pH 2 and extracted with EtOAc (2×10 mL). The organic layers were combined, dried (MgSO₄), filtered, and evaporated to give a white residue which was purified by HPLC. The desired fractions were pooled and lyophilized to yield 25 mg of Compound 1n as its TFA salt; MS(ES⁺) 662.

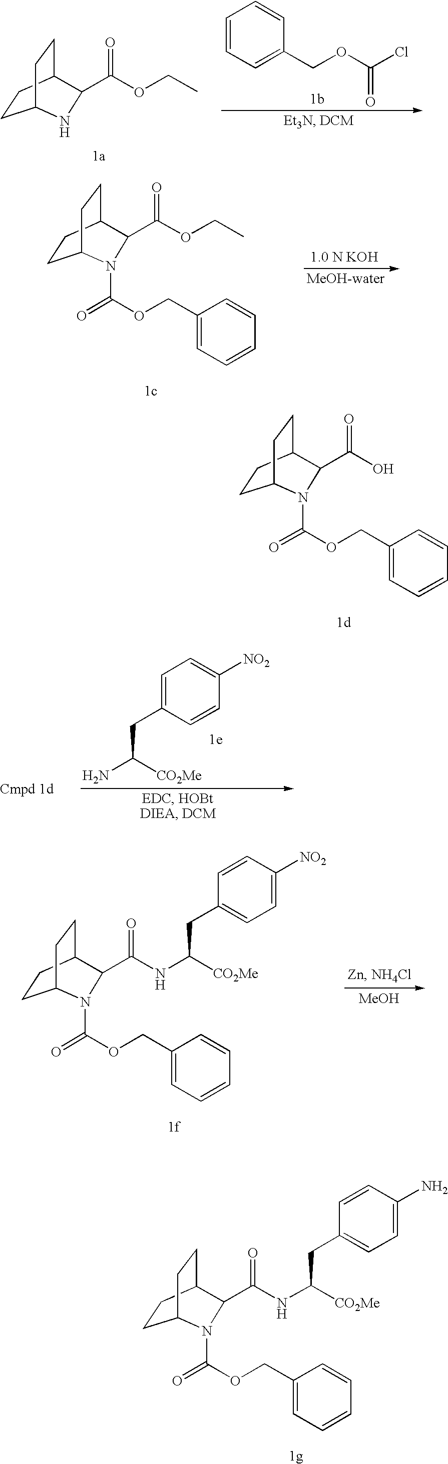

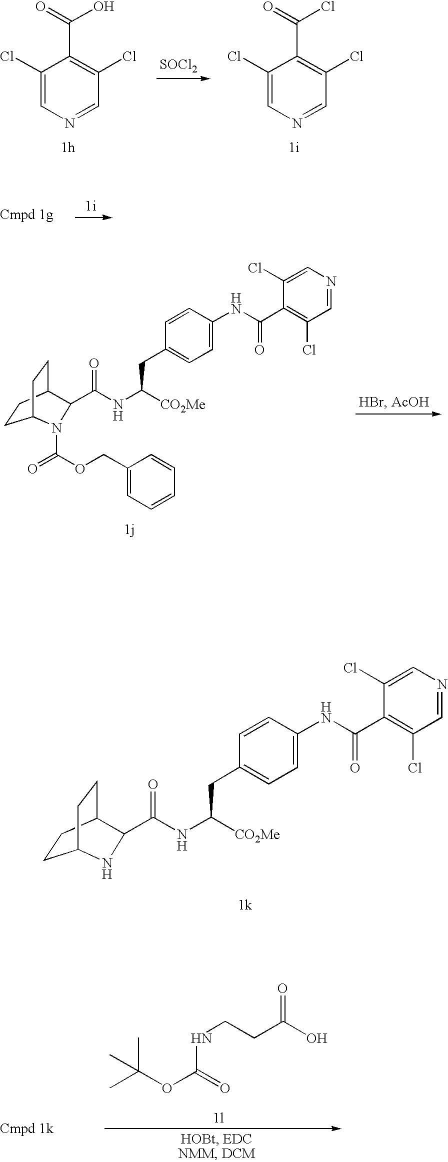

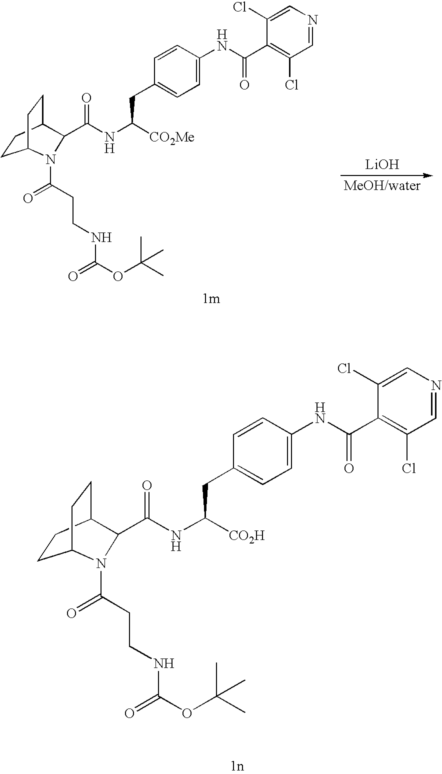

### EXAMPLE 2

To a solution of 2.6 g (8.9 mmol) of Compound 1d in DCM (100 mL) was added Compound 2a (2.1 g, 9 mmol), HOBt (1.6 g, 12 mmol), EDC (3.1 g, 16 mmol) and DIEA (4.7 mL, 27 mol). The resulting mixture was allowed to stir at rt overnight. The mixture was washed with a 10% citric acid solution, then by saturated aqueous NaHCO₃. The organic material was dried (MgSO₄) and concentrated to give 4.6 g of Compound 2b as a white foam which was used in the next reaction without further purification. LC 45%; MS(ES+) 467.

To a solution of Compound 2b (4.1 g, 8.9 mmol) in DCM (20 mL) was added Compound 2c (1.6 mL, 13 mmol) followed by Et₃N (2.5 mL, 17.8 mmol) and DMAP (0.5 g, 4.4 mmol). The mixture was allowed to stir at rt under argon for 5 h. LC analysis indicated the reaction was complete. The mixture was washed with 10% citric acid followed by saturated NaHCO₃ solution. The organic layer was dried (MgSO₄) and concentrated to 6.0 g of a yellow oil. The oil was purified (silica gel, 60:40, EtOAc:hexanes) to yield Compound 2d (2.0 g, 3.5 mmol, 39%) as a white tacky foam: LC 100%; TLC (80% EtOAc:hexanes) Rf=0.41; MS(ES+) 580.

To a solution of Compound 2d (1.9 g, 3.3 mmol) in EtOH was added 10% Pd/C (8 mg, 2 mol %). The resulting mixture was placed in a Parr hydrogenation apparatus with H₂ (40 psi) overnight. The mixture was filtered through Celite and the filtrate was concentrated. The concentrate was dissolved in DCM and washed with water followed by dilute HCl. The acidic aqueous layer was separated, basified with 1 N NaOH, and extracted with DCM (2×20 mL). The combined extracts were dried (MgSO₄) and concentrated to yield a clear oil (0.7 g). The original organic layer was found by LC analysis to contain additional product. The organic phase was washed again with dilute HCl and the aqueous phase was basified with 1 N NaOH, extracted with EtOAc, dried (MgSO₄) and concentrated to a clear oil (0.19 g). The individual crops were combined to give Compound 2e (0.89 g, 2 mmol, 67%) which was used for the next reaction without further purification. MS(ES+) 446.

Compound 2e (89 mg, 0.2 mmol), 3-(3,4-methylenedioxyphenyl)propionic acid 2f (42 mg, 0.216 mmol), EDC hydrochloride (42 mg, 0.22 mmol), and HOBt (8 mg, 0.21 mmol) were suspended in DCM (2 mL) at rt and N-methyl-morpholine (13.2 mL, 0.120 mmol) was added in one portion. The reaction was kept at rt for 4 h, evaporated and loaded into silica column. Flash chromatography (silica gel, EtOAc) provided 40 mg (59%) of Compound 2g as a white solid: MS(ES⁺) 622.

Compound 2g (40 mg, 0.06 mmol) was dissolved in a solution of MeOH/water (4 mL, 5/1) and LiOH—H₂O (4 mg, 0.1 mmol) was added as one portion. The resulting mixture was allowed to stir at rt overnight. The mixture was acidified with several drops of TFA, evaporated in vacuum and the residue was subjected to prep HPLC. Compound 2f (23 mg, 0.037 mmol, was obtained as a white powder: MS(ES⁺) 607.

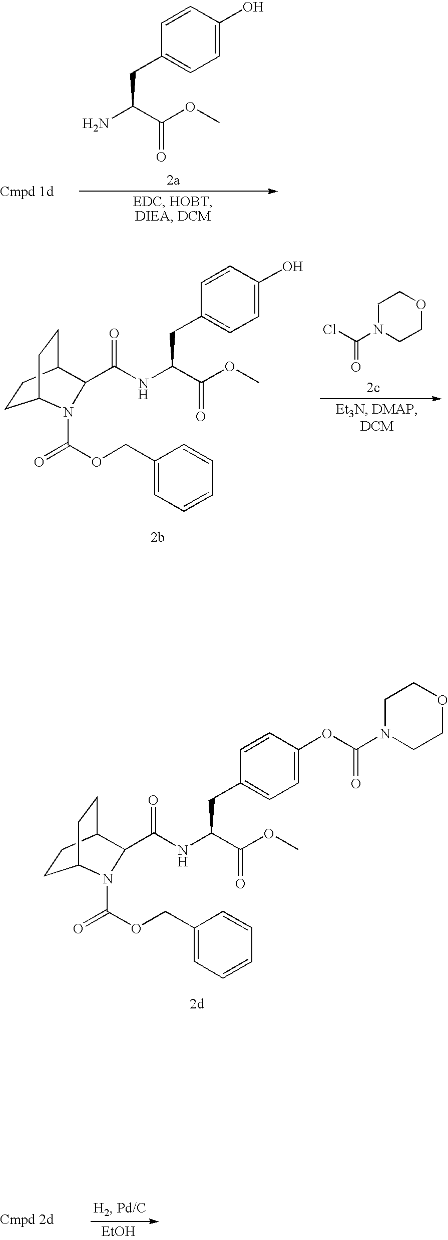

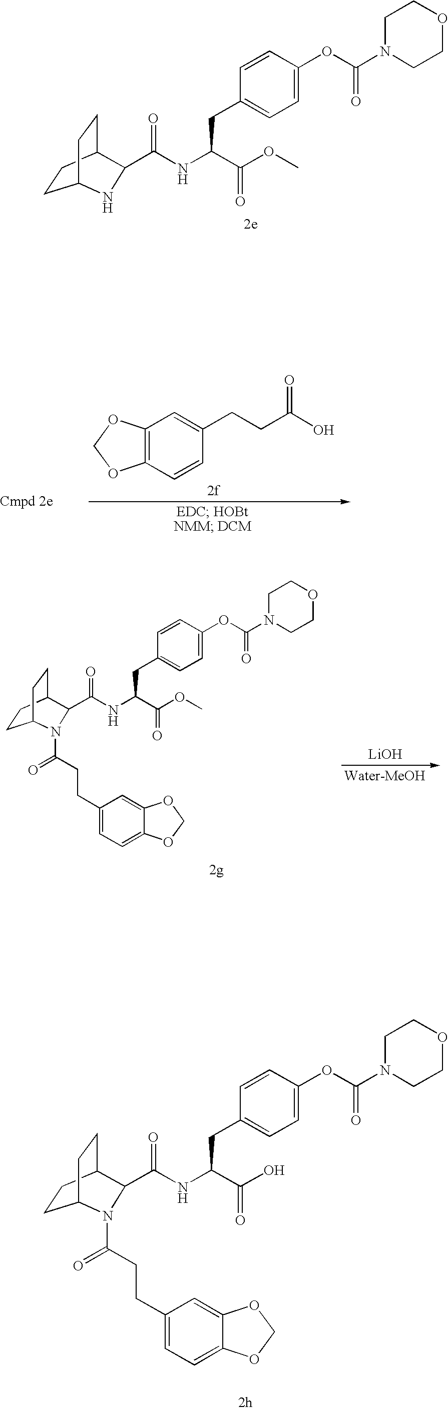

### Biological Experimental Examples

As demonstrated by biological studies described hereinafter, and shown in Table III and Table IV, the compounds of the present invention are α4β1 and α4β7 integrin receptor antagonists useful in treating integrin mediated disorders including, but not limited to, inflammatory, autoimmune and cell-proliferative disorders.

Ramos Cell Adhesion Assay (α₄β₁ Mediated Adhesion/VCAM-1) Immulon 96 well plates (Dynex) were coated with 100 μL recombinant hVCAM-1 at 4.0 μg/mL in 0.05 M NaCO₃ buffer pH 9.0 overnight at 4° C. (R&D Systems). Plates were washed 3 times in PBS with 1% BSA and blocked for 1 h @ room temperature in this buffer. PBS was removed and compounds to be tested (50 μL) were added at 2× concentration. Ramos cells, (50 μL at 2×10⁶/mL) labeled with 5 μM Calcein AM (Molecular Probes) for 1 h at 37° C., were added to each well and allowed to adhere for 1 h at room temperature. Plates were washed 3× in PBS+1% BSA and cells were lysed for 15 minutes in 100 μL of 1 M Tris pH 8.0 with 1% SDS. The plate was read at 485 nm excitation and 530 nm emission.

α₄β₇-K562 Cell Adhesion Assay (α₄β₇ Mediated Adhesion/VCAM-1) Immulon 96 well plates (Dynex) were coated with 100 μL recombinant hVCAM-1 at 4.0 μg/mL in 0.05 M NaCO₃ buffer pH 9.0 overnight at 4° C. (R&D Systems). Plates were washed 3 times in PBS with 1% BSA and blocked for 1 h @room temperature in this buffer. PBS was removed and compounds to be tested (50 μL) were added at 2× concentration. A stable cell line of K562 cells expressing human α₄β₇, (50 μL at 2×10⁶/mL) labeled with 5 μM Calcein AM (Molecular Probes) for 1 h at 37° C., were added to each well and allowed to adhere for 1 h at room temperature. Plates were washed 3× in PBS+1% BSA and cells were lysed for 15 minutes in 100 μL of 1 M Tris pH 8.0 with 1% SDS. The plate was read at 485 nm excitation and 530 nm emission.

TABLE

| | α4β1 | α4β7 |
|---|---|---|
| Cpd | IC₅₀ (nM) | IC₅₀ (nM) |
| 1 | 9 | 10 |
| 2 | 6 | 20 |
| 3 | 6 | 6 |
| 4 | >5000 | 3340 |
| 5 | 17 | 32 |
| 6 | 15 | 27 |
| 7 | 9 | 30 |
| 8 | 4 | 6 |
| 9 | 135 | 67 |
| 10 | >1000 | 896 |
| 11 | 14 | 47 |
| 12 | 8 | 117 |
| 13 | >1000 | 2370 |
| 14 | 1270 | 304 |
| 15 | 29 | 60 |
| 16 | 23 | 177 |
| 17 | 104 | 417 |
| 18 | 121 | 42 |
| 19 | 68 | 172 |
| 20 | 137 | 437 |
| 21 | 2 | 3 |
| 22 | 45 | 35 |
| 23 | 217 | 277 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A compound of Formula (I): 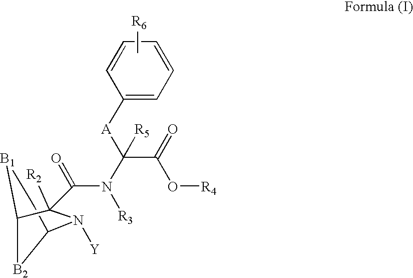
wherein
Y is selected from the group consisting of a hydrogen, —C(O)(CH₂)₀₋₄R₁₈, C(O)(CH₂)_{q}NC(O)R₁, C(O)(CH₂)_{q}SR₁, —C(O)(CH₂)_{q}SOR₁; and —C(O)(CH₂)_{q}SO₂R₁;
q is an integer from 1 to 8;
R₁ is selected from the group consisting of hydrogen, R₇ and R₈;
R₂, R₃, and R₅ are independently selected from the group consisting of hydrogen and C₁₋₈alkyl; wherein C₁₋₈alkyl is optionally substituted with one to three substituents independently selected from OH, halogen, C₁₋₈ alkoxy, carboxy, amino, N-(C₁₋₈ alkyl)amino, N,N-(C₁₋₈ dialkyl)amino, CF₃, OCF₃ and R₉; provided that R₃ additionally may be a bond when forming a monocyclic ring;
R₄ is selected from the group consisting of hydrogen, C₁₋₄alkyl and N,N-C₁₋₄ glycolamide; wherein C₁₋₄alkyl is optionally substituted with one to three substituents independently selected from OH, halogen, C₁₋₈ alkoxy, carboxy, amino, N-(C₁₋₈ alkyl)amino, N,N-(C₁₋₈ dialkyl)amino, CF₃, OCF₃ and R₉; provided that R₄ additionally may be a bond when forming a monocyclic ring;
wherein R₃ and R₄ may form a monocyclic ring
when R₃ and R₄ comprise a bond and C₁₋₈alkyl or optionally when both R₃ and R₄ are C₁₋₈alkyl, R₃ and R₄ together with the atoms to which each is attached will form a five to seven membered monocyclic ring optionally containing one to two additional heteroatoms independently selected from the group consisting of N, O and S;
R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀, —N(R₁₁)SO₂—R₁₂, —N(R₁₁)C(O)—N(R₁₁,R₁₀), —N(R₁₁)C(O)—N(R₁₁,R₁₂), —N(R₁₁)C(O)—N(R₁₂,R₁₇), —C(O)—N(R₁₁,R₁₀), —C(O)—N(R₁₁,R₁₂), —C(O)—N(R₁₂,R₁₇), —OC(O)—N(R₁₁,R₁₀), —OC(O)—N(R₁₁,R₁₂), —OC(O)—N(R₁₂,R₁₇), —OC(O)—R₁₀, —OC(O)—R₁₂, —O—R₁₀ and R₁₀-(C₁₋₈)alkoxy;
R₇, R₉ R₁₀ and R₁₄ are independently selected from the group consisting of cycloalkyl, heterocyclyl, aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
R₈ is selected from the group consisting of C₁₋₈alkyl, C₂₋₈alkenyl, C₂₋₈alkynyl, C₁₋₈alkoxy and (halo)₁₋₃(C₁₋₈)alkyl; wherein C₁₋₈alkyl, C₂₋₈alkenyl, C₂₋₈alkynyl and C₁₋₈alkoxy are optionally substituted with one to three substituents independently selected from R₁₄;
R₁₂, R₁₃, R₁₇ and R₁₉ are independently selected from the group consisting of C₁₋₈alkyl, C₂₋₈alkenyl, C₂₋₈alkynyl and (halo)₁₋₃(C₁₋₈)alkyl; wherein C₁₋₈alkyl, C₂₋₈alkenyl and C₂₋₈alkynyl are optionally substituted with one to three substituents independently selected from R₁₄;
R₁₈ is selected from the group consisting of hydroxy, C₁₋₈alkoxy, C₁₋₈ alkyloxyC₁₋₈alkyl, C₁₋₈ alkylcarbonyl aminoC₁₋₈ alkyl, amino, amino C₁₋₈alkyl, C₁₋₈alkyl amino C₁₋₈alkyl, diC₁₋₈alkylamino C₁₋₈alkyl, benzo-fused heterocyclyl, polycycloalkyl and hydroxy C₁₋₈alkyl; wherein the benzo-fused heterocyclyl is substituted with C(O)R₁₉ and C(O)OR₁₉.
R₁₁ is selected from the group consisting of hydrogen and C₁₋₈alkyl;
A is C₁₋₂alkylene optionally substituted with one to two substituents independently selected from R₁₃;
when R₃ is C₁₋₈alkyl, optionally A and R₃ together with the atoms to which each is attached may form a five to seven membered monocyclic ring optionally containing one to two additional heteroatoms independently selected from the group consisting of N, O and S;
when R₄ is C₁₋₈alkyl, optionally A and R₄ together with the atoms which each is attached may form a five to seven membered monocyclic ring optionally containing one additional heteroatom selected from the group consisting of N, O and S;
when R₅ is C₁₋₈alkyl, optionally A and R₅ together with the atoms which each is attached may form a three to seven membered monocyclic ring optionally containing one to two heteroatoms independently selected from the group consisting of N, O and S; and,
B₁ and B₂ are independently selected from the group consisting of C₁₋₂alkylene and C₂alkenylene optionally substituted with one to two substituents independently selected from the group consisting of halogen, hydroxy, hydroxy(C₁₋₈)alkyl, hydroxy(C₁₋₈)alkoxy, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
and pharmaceutically acceptable salts, racemic mixtures, diastereomers and enantiomers thereof.

2. The compound of claim 1 wherein Y is hydrogen.

3. The compound of claim 1 wherein Y is selected from the group consisting of —C(O)R₁₈, C(O)(CH₂)_{q}NC(O)R₁, —C(O)(CH₂)_{q}SR₁, —C(O)(CH₂)_{q}SOR₁; and —C(O)(CH₂)_{q}SO₂R₁, wherein q is an integer from 1 to 4.

4. The compound of claim 3 wherein q is an integer from 1 to 2.

5. The compound of claim 1 wherein R₁₈ is selected from the group consisting of of hydroxy, C₁₋₄alkoxy, C₁₋₈ alkyloxyC₁₋₈alkyl, C₁₋₈ alkylcarbonyl aminoC₁₋₈ alkyl, amino C₁₋₄alkyl, C₁₋₄alkyl amino C₁₋₄alkyl, diC₁₋₄alkylamino C₁₋₄alkyl, benzo-fused heterocyclyl, C₁₀polycycloalkyl and hydroxy C₁₋₄alkyl; wherein the benzo-fused heterocyclyl is substituted with C(O)R₁₉ and C(O)OR₁₉.

6. The compound of claim 1 wherein R₁₉ is selected from the group consisting of C₁₋₄alkyl, C₂₋₄alkenyl, C₂₋₄alkynyl, and (halo)₁₋₃(C₁₋₄)alkyl; wherein C₁₋₄alkyl, C₂₋₈alkenyl and C₂₋₄alkynyl are optionally substituted on a terminal carbon with one to three substituents independently selected from R₁₄.

7. The compound of claim 1 wherein R₁ is R₇.

8. The compound of claim 1 wherein R₂, R₃, R₄ and R₅ are independently selected from the group consisting of hydrogen and C₁₋₄alkyl.

9. The compound of claim 1 wherein R₂, R₃, R₄ and R₅ are independently selected from the group consisting of hydrogen and methyl.

10. The compound of claim 1 wherein R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀—, —N(R₁₁)C(O)—N(R₁₁,R₁₂), —N(R₁₁)C(O)—N(R₁₂,R₁₇), —OC(O)—N(R₁₁,R₁₂), —OC(O)—N(R₁₂,R₁₇), —OC(O)—R₁₀ and R₁₀—(C₁₋₈)alkoxy.

11. The compound of claim 1 wherein R₆ is optionally present and is one to three substituents independently selected from the group consisting of halogen, C₁₋₄alkoxy, R₁₀, R₁₂, —N(R₁₁)C(O)—R₁₀, —N(R₁₁)C(O)—R₁₂, —N(R₁₁)SO₂—R₁₀—, —N(R₁₁)C(O)—N(R₁₁,R₁₂), —N(R₁₁)C(O)—N(R₁₂,R₁₇), —OC(O)—N(R₁₁,R₁₂), —OC(O)—N(R₁₂,R₁₇), —OC(O)—R₁₀ and R₁₀—(C₁₋₄)alkoxy.

12. The compound of claim 1 wherein R₆ is optionally present and is one to two substituents independently selected from the group consisting of R₁₀, —N(R₁₁)C(O)—R₁₀, —N(R₁₁)C(O)—N(R₁₁,R₁₂), —N(R₁₁)C(O)—N(R₁₂,R₁₇), —OC(O)—N(R₁₂,R₁₂), —OC(O)—N(R₁₂,R₁₇)—OC(O)—R₁₀ and R₁₀-methoxy.

13. The compound of claim 1 wherein R₇ is selected from the group consisting of aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃.

14. The compound of claim 1 wherein R₁₀ is selected from the group consisting of cycloalkyl, heterocyclyl, aryl and heteroaryl optionally substituted with one to five substituents independently selected from the group consisting of halogen, C₁₋₈alkyl, C₁₋₈alkoxy, C₁₋₈alkoxycarbonyl, carboxyl, arylcarbonyl, arylsulfonyl, —CF₃ and —OCF₃; wherein cycloalkyl and heterocyclyl are optionally substituted with one to three oxo substituents; and, wherein the aryl portion of the arylcarbonyl substituent is optionally substituted with one to five substituents independently selected from C₁₋₈alkoxy.

15. The compound of claim 1 wherein R₁₀ is selected from the group consisting of cyclopropyl, 1,3-dihydro-2H-isoindolyl, 2-azabicyclo[2.2.2]octyl, piperidinyl, morpholinyl, phenyl, naphthalenyl, thienyl, 1H-pyrrolyl and pyridinyl; wherein cyclopropyl, piperidinyl, morpholinyl, phenyl, naphthalenyl, thienyl, 1H-pyrrolyl and pyridinyl are optionally substituted with one to four substituents independently selected from the group consisting of chlorine, fluorine, bromine, methyl, isopropyl, t-butyl, methoxy, t-butoxycarbonyl, carboxyl, phenylcarbonyl, —CF₃ and —OCF₃; wherein 1,3-dihydro-2H-isoindolyl is optionally substituted with oxo; wherein 2-azabicyclo[2.2.2]octyl is optionally substituted with phenylsulfonyl, and, wherein the phenyl portion of the phenylcarbonyl substituent is optionally substituted with one to two substituents independently selected from methoxy.

16. The compound of claim 1 wherein R₁₂ is selected from the group consisting of C₁₋₈alkyl and C₂₋₈alkynyl optionally substituted with R₁₄.

17. The compound of claim 1 wherein R₁₂ is selected from the group consisting of C₁₋₄alkyl and C₂₋₄alkynyl optionally substituted with R₁₄.

18. The compound of claim 1 wherein R₁₂ is selected from the group consisting of t-butyl and ethynyl; wherein ethynyl is optionally substituted with a substituent independently selected from R₁₄.

19. The compound of claim 1 wherein R₁₄ is selected from the group consisting of aryl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, arylcarbonyl, heteroarylcarbonyl, arylsulfonyl, amino, N-(C₁₋₈alkyl)amino, N,N-(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃.

20. The compound of claim 1 wherein R₁₁, is selected from the group consisting of hydrogen and C₁₋₄alkyl.

21. The compound of claim 1 wherein R₁₁, is hydrogen.

22. The compound of claim 1 wherein A is selected from the group consisting of methylene and ethylene.

23. The compound of claim 1 wherein B₁ and B₂ are independently selected from the group consisting of C₁₋₂alkylene and C₂alkenylene optionally substituted with one to two substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃.

24. The compound of claim 1 wherein B₁ and B₂ are independently selected from the group consisting of —CH₂—, —(CH₂)₂— and —(CH)₂— optionally substituted with one to substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃.

25. The compound of claim 1 wherein B₁ is selected from the group consisting of —CH₂—, —(CH₂)₂— and —(CH)₂— optionally substituted with one to two substituents independently selected from the group consisting of halogen, hydroxy, hydroxy(C₁₋₄)alkyl, hydroxy(C₁₋₄)alkoxy, C₁₋₄alkyl, C₂₋₄alkenyl, C₂₋₄alkynyl, C₁₋₄alkoxy, carboxyl, amino, N-(C₁₋₄alkyl)amino, N,N-(C₁₋₄dialkyl)amino, —CF₃ and —OCF₃; and, wherein, B₂ is selected from —(CH₂)₂—.

26. The compound of claim 1 wherein B₁ is selected from the group consisting of —CH₂—, —(CH₂)₂— and —(CH)₂—.

27. A compound having Formula (III): 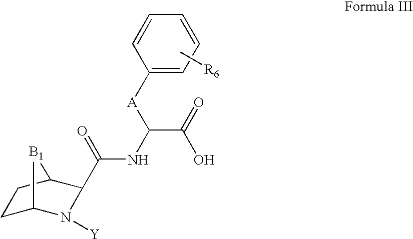
wherein
Y is selected from the group consisting of a bond, —C(O)R₁₈, C(O)(CH₂)_{q}NC(O)R₁, —C(O)(CH₂)_{q}SR₁, —C(O)(CH₂)_{q}SOR₁; and —C(O)(CH₂)_{q}SO₂R₁;
q is an integer from 1 to 8;
R₁ is selected from the group consisting of hydrogen, R₇ and R₈;
R₂, R₃, and R₅ are independently selected from the group consisting of hydrogen and C₁₋₈alkyl; wherein C₁₋₈alkyl is optionally substituted with one to three substituents independently selected from OH, halogen, C₁₋₈ alkoxy, carboxy, amino, N-(C₁₋₈ alkyl)amino, N,N-(C₁₋₈ dialkyl)amino, CF₃, OCF₃ and R₉; provided that R₃ additionally may be a bond when forming a monocyclic ring;
R₄ is selected from the group consisting of hydrogen, C₁₋₄alkyl and N,N-C₁₋₄ glycolamide; wherein C₁₋₄alkyl is optionally substituted with one to three substituents independently selected from OH, halogen, C₁₋₈ alkoxy, carboxy, amino, N-(C₁₋₈ alkyl)amino, N,N-(C₁₋₈ dialkyl)amino, CF₃, OCF₃ and R₉; provided that R₄ additionally may be a bond when forming a monocyclic ring;
wherein R₃ and R₄ may form a monocyclic ring
when R₃ and R₄ comprise a bond and C₁₋₈alkyl or optionally when both R₃ and R₄ are C₁₋₈alkyl, R₃ and R₄ together with the atoms to which each is attached will form a five to seven membered monocyclic ring optionally containing one to two additional heteroatoms independently selected from the group consisting of N, O and S;
R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀, —N(R₁₁)SO₂—R₁₂, —N(R₁₁)C(O)—N(R₁₁,R₁₀), —N(R₁₁)C(O)—N(R₁₁,R₁₂), —N(R₁₁)C(O)—N(R₁₂,R₁₇), —C(O)—N(R₁₁,R₁₀), —C(O)—N(R₁₁,R₁₂), —C(O)—N(R₁₂,R₁₇), —OC(O)—N(R₁₁,R₁₀), —OC(O)—N(R₁₁,R₁₂), —OC(O)—N(R₁₂,R₁₇), —OC(O)—R₁₀, —OC(O)—R₁₂, —O—R₁₀ and R₁₀-(C₁₋₈)alkoxy;
R₇, R₉ R₁₀ and R₁₄ are independently selected from the group consisting of cycloalkyl, heterocyclyl, aryl, heteroaryl, benzo-fused heterocyclyl and benzo-fused cycloalkyl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, aryloxy, heteroaryloxy, cycloalkyloxy, heterocycloxy, benzyloxy carbonyl, arylcarbonyl, heteroarylcarbonyl, arylsulfonyl, amino, N-(C₁₋₈alkyl)amino, N,N-(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
R₈ is selected from the group consisting of C₁₋₈alkyl, C₂₋₈alkenyl, C₂₋₈alkynyl, C₁₋₈alkoxy and (halo)₁₋₃(C₁₋₈)alkyl; wherein C₁₋₈alkyl, C₂₋₈alkenyl, C₂₋₈alkynyl and C₁₋₈alkoxy are optionally substituted with one to three substituents independently selected from R₁₄;
R₁₂, R₁₃, R₁₇ and R₁₉ are independently selected from the group consisting of C₁₋₈alkyl, C₂₋₈alkenyl, C₂₋₈alkynyl and (halo)₁₋₃(C₁₋₈)alkyl; wherein C₁₋₈alkyl, C₂₋₈alkenyl and C₂₋₈alkynyl are optionally substituted with one to three substituents independently selected from R₁₄;
R₁₈ is selected from the group consisting of hydroxy, C₁₋₈alkoxy, C₁₋₈ alkyloxyC₁₋₈alkyl, C₁₋₈ alkylcarbonyl aminoC₁₋₈ alkyl, amino, amino C₁₋₈alkyl, C₁₋₈alkyl amino C₁₋₈alkyl, diC₁₋₈alkylamino C₁₋₈alkyl, benzo-fused heterocyclyl, polycycloalkyl and hydroxy C₁₋₈alkyl; wherein the benzo-fused heterocyclyl is substituted with C(O)R₁₉ and C(O)OR₁₉.
R₁₁ is selected from the group consisting of hydrogen and C₁₋₈alkyl;
A is C₁₋₂alkylene optionally substituted with one to two substituents independently selected from R₁₃;
when R₃ is C₁₋₈alkyl, optionally A and R₃ together with the atoms to which each is attached may form a five to seven membered monocyclic ring optionally containing one to two additional heteroatoms independently selected from the group consisting of N, O and S;
when R₄ is C₁₋₈alkyl, optionally A and R₄ together with the atoms which each is attached may form a five to seven membered monocyclic ring optionally containing one additional heteroatom selected from the group consisting of N, O and S;
when R₅ is C₁₋₈alkyl, optionally A and R₅ together with the atoms which each is attached may form a three to seven membered monocyclic ring optionally containing one to two heteroatoms independently selected from the group consisting of N, O and S; and,
B₁ and B₂ are independently selected from the group consisting of C₁₋₂alkylene and C₂alkenylene optionally substituted with one to two substituents independently selected from the group consisting of halogen, hydroxy, hydroxy(C₁₋₈)alkyl, hydroxy(C₁₋₈)alkoxy, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
and pharmaceutically acceptable salts, racemic mixtures, diastereomers and enantiomers thereof.

28. The compound of claim 27, wherein B₁, Y, R₁, A and R₆ are selected from the group consisting of:

| B₁ | Y | R₁ | A | R₆ |
|---|---|---|---|---|
| (CH₂)₂ | C(O)(CH₂)S R₁ | 2-pyridinyl | CH₂ | 4-NHC(O)-(3,5- |
| | | | | Cl₂)pyridinyl |
| (CH₂)₂ | C(O)(CH₂)S R₁ | -(4,F)phenyl | CH₂ | 4-NHC(O)-(3,5- |
| | | | | Cl₂)pyridinyl |
| (CH₂)₂ | C(O)(CH₂)S R₁ | 4-pyridinyl | CH₂ | 4-NHC(O)-(3,5- |
| | | | | Cl₂)pyridinyl |
| (CH₂)₂ | C(O)(CH₂)SO₂ R₁ | —CH₃ | CH₂ | 4-NHC(O)-(3,5- |
| | | | | Cl₂)pyridinyl |
| (CH₂)₂ | C(O)(CH₂)SO₂ R₁ | -phenyl | CH₂ | 4-NHC(O)-(3,5- |
| | | | | Cl₂)pyridinyl |
| (CH₂)₂ | C(O)(CH₂)₂NC(O) R₁ | —OC(CH₃)₃ | CH₂ | 4-NHC(O)-(3,5- |
| | | | | Cl₂)pyridinyl |

and pharmaceutically acceptable salts, racemic mixtures, diastereomers and salts thereof.

29. A compound of Formula (IV) 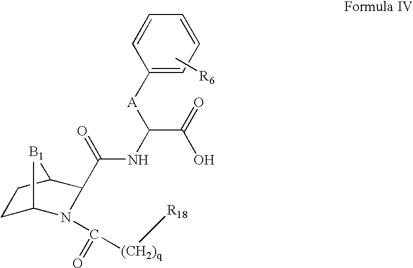
wherein
q is an integer from 1 to 8;
R₆ is optionally present and is one to three substituents independently selected from the group consisting of halogen, C₁₋₈alkoxy, R₁₀, R₁₂, —N(R₁₁)C(O)—R₁₀, —N(R₁₁)C(O)—R₁₂, —N(R₁₁)SO₂—R₁₀, —N(R₁₁)SO₂—R₁₂, —N(R₁₁)C(O)—N(R₁₁,R₁₀), —N(R₁₁)C(O)—N(R₁₁,R₁₂), —N(R₁₁)C(O)—N(R₁₂,R₁₇), —C(O)—N(R₁₁,R₁₀), —C(O)—N(R₁₁,R₁₂), —C(O)—N(R₁₂,R₁₇), —OC(O)—N(R₁₁,R₁₀), —OC(O)—N(R₁₁,R₁₂), —OC(O)—N(R₁₂,R₁₇), —OC(O)—R₁₀, —OC(O)—R₁₂, —O—R₁₀ and R₁₀-(C₁₋₈)alkoxy;
R₁₀ and R₁₄ are independently selected from the group consisting of cycloalkyl, heterocyclyl, aryl and heteroaryl optionally substituted with one to five substituents independently selected from the group consisting of halogen, C₁₋₈alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, arylcarbonyl, heteroarylcarbonyl, arylsulfonyl, amino, N-(C₁₋₈alkyl)amino, N,N-(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
R₁₁ is selected from the group consisting of hydrogen and C₁₋₈alkyl;
R₁₂, R₁₃, R₁₇ and R₁g are independently selected from the group consisting of C₁₋₈alkyl, C₂₋₈alkenyl, C₂₋₈alkynyl, and (halo)₁₋₃(C₁₋₈)alkyl; wherein C₁₋₈alkyl, C₂₋₈alkenyl and C₂₋₈alkynyl are optionally substituted with one to three substituents independently selected from R₁₄;
R₁₈ is selected from the group consisting of hydroxy, C₁₋₈alkoxy, C₁₋₈ alkyloxyC₁₋₈alkyl, C₁₋₈ alkylcarbonyl aminoC₁₋₈ alkyl, amino, amino C₁₋₈alkyl, C₁₋₈alkyl amino C₁₋₈alkyl, diC₁₋₈alkylamino C₁₋₈alkyl, benzo-fused heterocyclyl, polycycloalkyl and hydroxy C₁₋₈alkyl; wherein the benzo-fused heterocyclyl is substituted with C(O)R₁₉ and C(O)OR₁₉.
A is C₁₋₂alkylene optionally substituted with one to two substituents independently selected from R₁₃;
B₁ is selected from the group consisting of C₁₋₈alkylene and C₂₋₈alkenylene optionally substituted with one to two substituents independently selected from the group consisting of halogen, hydroxy, hydroxy(C₁₋₈)alkyl, hydroxy(C₁₋₈)alkoxy, C₁₋₈alkyl, C₂₋₈alkenyl, C₂₋₈alkynyl, C₁₋₈alkoxy, carboxyl, amino, N-(C₁₋₈alkyl)amino, N,N-(C₁₋₈dialkyl)amino, —CF₃ and —OCF₃;
and pharmaceutically acceptable salts, racemic mixtures, diastereomers and enantiomers thereof.

30. The compound of claim 29 wherein B₁, q, R₁₈, A and R₆ are selected from the group consisting of:

| B₁ | q | R₁₈ | A | R₆ |
|---|---|---|---|---|
| (CH₂)₂ | 2 | —OH | CH₂ | 4-NHC(O)-(3,5- |
| | | | | Cl₂)pyridin-4-yl |
| (CH₂)₂ | 2 | —OH | CH₂ | 4-NHC(O)-(2,6- |
| | | | | Cl₂)phenyl |
| (CH₂)₂ | 2 | CH₂C(CH₃)₂OH | CH₂ | 4-NHC(O)-(3,5- |
| | | | | Cl₂)pyridin-4-yl |
| (CH₂)₂ | 2 | —OCH₃ | CH₂ | 4-NHC(O)-(3,5- |
| | | | | Cl₂)pyridin-4-yl |
| (CH₂)₂ | 2 | —OCH₃ | CH₂ | 4-OC(O)N(CH₃)₂ |
| (CH₂)₂ | 2 | —NH₂ | CH₂ | 4-NHC(O)-(3,5- |
| | | | | Cl₂)pyridin-4-yl |
| (CH₂)₂ | 2 | N(CH₃)₂ | CH₂ | 4-NHC(O)-(3,5- |
| | | | | Cl₂)pyridin-4-yl |
| (CH₂)₂ | 2 | Adamantyl | CH₂ | 4-NHC(O)-(3,5- |
| | | | | Cl₂)pyridin-4-yl |
| (CH₂)₂ | 1 | 2,3,4,5-tetrahydro-benzo | CH₂ | 4-NHC(O)-(3,5- |
| | | [f]-[1,4]oxazepine | | Cl₂)pyridin-4-yl |
| (CH₂)₂ | 1 | 3,4,4a,8a-tetrahydro-1H- | | 4-NHC(O)-(3,5- |
| | | isoquinoline | | Cl₂)pyridin-4-yl |
| (CH₂)₂ | 1 | 3,4,4a,8a-tetrahydro-1H- | CH₂ | 4-NHC(O)-(3,5- |
| | | isoquinoline-2-tert- | | Cl₂)pyridin-4-yl |
| | | butoxycarbonyl | | |
| (CH₂)₂ | 1 | 5-benzyl[1,3]dioxole | CH₂ | 4-NHC(O)-(3,5- |
| | | | | Cl₂)pyridin-4-yl |
| (CH₂)₂ | 2 | 5-benzyl[1,3]dioxole | CH₂ | 4-NHC(O)-(2,6- |
| | | | | Cl₂)phenyl |
| (CH₂)₂ | 2 | 5-benzyl[1,3]dioxole | CH₂ | 4-OC(O)-morpholin-1- |
| | | | | yl |

and pharmaceutically acceptable salts, racemic mixtures, diastereomers and enantiomers thereof.

31. The compound of claim 1 wherein the compounds are effective antagonists of an integrin receptor.

32. The compound of claim 31 wherein the compound is a selective antagonist of an α4 integrin receptor.

33. The compound of claim 32 wherein the α4 integrin receptor is selected from the group consisting of the α4β1 and α4β7 integrin receptor.

34. The compound of claim 31 wherein the compound is an antagonist of at least two α4 integrin receptors.

35. The compound of claim 34 wherein the two α4 integrin receptors are selected from the group consisting of the α4β1 and α4β7 integrin receptor.

36. The compound of claim 1 wherein the compounds are effective agents for the treatment of integrin mediated disorder selected from the group consisting of inflammatory disorders, autoimmunde disorders and cell-proliferative disorders.

37. The compound of claim 36 wherein the integrin mediated disorder is selected from the group consisting of inflammation disorders, autoimmunity disorders, asthma, bronchoconstriction, restenosis, atherosclerosis, psoriasis, rheumatoid arthritis, inflammatory bowel disease, irritable bowel disease, irritable bowel syndrome, transplant rejection and multiple sclerosis.

38. The compound of claim 36 wherein the integrin mediated disorder is selected from the group consisting of asthma, bronchoconstriction, restenosis, atherosclerosis, psoriasis, rheumatoid arthritis, inflammatory bowel disease, irritable bowel disease, irritable bowel syndrome, transplant rejection and multiple sclerosis.

39. The compound of claim 36 wherein the integrin mediated disorder is selected from the group consisting of asthma, bronchoconstriction, restenosis, atherosclerosis, irritable bowel syndrome and multiple sclerosis.

40. A pharmaceutical composition comprising a compound of claim 1 and a pharmaceutically acceptable carrier.

41. A method for the treatment of an integrin mediated disorder ameliorated by inhibition of an α4 integrin receptor comprising administering to a subject in need thereof a therapeutically effective amount of a compound of claim 1.

42. The method of claim 41 wherein the compound inhibiting the α4 integrin receptor is selected from the group consisting of a selective antagonist of the α4β1 integrin receptor, a selective antagonist of the α4β7 integrin receptor and an antagonist of the α4β1 and α4β7 integrin receptors.

43. The method of claim 1 wherein the therapeutically effective amount of the compound of claim 1 is from about 0.01 mg/kg/day to about 300 mg/kg/day.

44. The compound of claim 1 wherein R₇ is selected from the group consisting tolyl, phenyl and thienyl.
